(19) United States
(12) Patent Application Publication
(10) Pub. No.: US 2007/0031361 A1
(43) Pub. Date: Feb. 8, 2007

(54) Cosmetic, pharmaceutical and dermatological preparations comprising homopolymer and/or copolymer waxes of the monomers ethylene and/or propylene

(76) Inventors: Hans-Friedrich Herrmann, Gross-Gerau (DE); Anton Lukasch, Meitingen (DE); Gerd Hohner, Gersthofen (DE); Heike Michaelis, Darmstadt (DE); Angela Lachmann, Kelkheim-Fischbach (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/449,051

(22) Filed: Jun. 8, 2006

(30) Foreign Application Priority Data
Aug. 6, 2005 (DE) 102005026278.3

(51) Int. Cl. A61K 8/81

(52) U.S. Cl. 424/70.11

(57) ABSTRACT

Cosmetic, pharmaceutical or dermatological preparations are described which comprise one or more homopolymer and/or copolymer waxes of the monomers ethylene and/or propylene. The homopolymer and copolymer waxes have a weight-average molecular weight Mw of less than or equal to 25 000 g/mol, a number-average molecular weight Mn of less than or equal to 15 000 g/mol, a molar mass distribution Mw/Mn in the range from 1.5 to 10 and have been obtained by metallocene catalysis. The copolymer waxes comprise, based on the total weight of the copolymer waxes, 0.1 to 30.0% by weight of structural units originating from the one monomer and 70.0 to 99.9% by weight of structural units originating from the other monomer.

The present invention is described in the German priority application No. 102005026278.3, filed Aug. 6, 2005, which is hereby incorporated by reference as is fully disclosed herein.

The invention relates to cosmetic, pharmaceutical and dermatological preparations comprising homopolymer and/or copolymer waxes obtained by low-pressure polymerization of ethylene and/or propylene in the presence of a metallocene catalyst.

Waxes and wax-like substances primarily determine the consistency of many cosmetic products. Waxes are used in order to influence the hardness and solidity of cosmetic products. The more particularly hard waxes which only melt at high temperatures are used, the more solid the product. Waxes are also advantageously used in liquid, cream-like and gel-like preparations. They improve the water resistance and the cohesion of the compositions on skin and hair, are odor- and taste-neutral and can be readily processed.

In cosmetics, natural waxes of animal and vegetable origin, such as beeswax, berry wax, rose wax, japan wax, china wax, shellac wax, quince wax, shea butter, candelilla wax, carnauba wax, lanolin (wool fat), jojoba oil and jojoba wax, are often used. Because natural waxes are contaminated with pesticides, alternative, synthetically produced waxes are sought which are free from crop protection compositions and allergenic substances. Moreover, wax-like substances are sought which exhibit good skin sensory properties, have good film formation and film stability and are compatible with customary cosmetic ingredients.

DE 100 64 799 describes the use of ethylene polymer waxes and ethylene copolymer waxes, produced under high-pressure conditions, in cosmetic preparations. In this process, aliphatic and alicyclic ketones are used as molecular weight regulators in order to obtain ethylene (co)polymer waxes with suitable molecular weights. Ketones can have a sensitizing effect and are undesired in cosmetic formulations.

WO 2002/049592 describes antiperspirants and deodorants which, besides cyclomethicones and active ingredients, comprise low molecular weight, predominantly linear polyethylene waxes with molecular weights of from 300 to 3000 g/mol, preferably 300 to 500 g/mol, for example Performalene®. These low molecular weight polyethylene waxes are brittle, not very elastic and have low adhesion to skin and hair.

It was the object to provide substances for cosmetic, pharmaceutical and dermatological preparations which have similarly good consistency-imparting properties as naturally occurring waxes, are readily compatible with aqueous systems and with oil systems, have a clear visual appearance, are readily processible and compatible with active substances (e.g. sunscreen filters), but are also skin-compatible and toxicologically acceptable, have good skin sensory properties, adhere well to skin and hair and are water-resistant.

Surprisingly, it has been found that this object is achieved by certain homopolymer and/or copolymer waxes of the monomers ethylene and/or propylene with a weight-average molecular weight Mw of less than or equal to 25 000 g/mol and a narrow molecular weight distribution produced by low-pressure polymerization of ethylene and/or propylene in the presence of a metallocene catalyst.

The invention provides cosmetic, pharmaceutical and dermatological preparations comprising one or more homopolymer and/or copolymer waxes of the monomers ethylene and/or propylene, wherein the homopolymer and copolymer waxes have a weight-average molecular weight Mw of less than or equal to 25 000 g/mol, a number-average molecular weight Mn of less than or equal to 15 000 g/mol, a molar mass distribution Mw/Mn in the range from 1.5 to 10, preferably from 1.5 to 5, particularly preferably from 1.5 to 3 and especially preferably from 2 to 2.5, and have been obtained by metallocene catalysis and where the copolymer waxes, based on the total weight of the copolymer waxes, comprise 0.1 to 30.0% by weight of structural units originating from the one monomer and 70.0 to 99.9% by weight of structural units originating from the other monomer.

These homopolymer and/or copolymer waxes have a white appearance, are odor- and taste-neutral, readily processible and highly suited to the shape-stabilization of solid compositions, for example of sticks. They are suitable for setting the viscosity of creamy emulsions or dispersions, and also compositions containing hydroxy acid and electrolyte and can be processed to give flowable preparations. They significantly improve the absorption capacity of pigments in the lipid phase and the pigment dispersion, and also the effect of effect pigments. The water resistance and adhesive strength of the compositions is very advantageous for the cosmetic application. The migration of solid ingredients (e.g. pigments) is suppressed, as is the tendency of individual ingredients to penetrate into the skin. A reduction in the irritancy effect of ingredients is thus achieved. The homopolymer and/or copolymer waxes can advantageously be used as active ingredient carrier matrix for a targeted and delayed release of active substances.

The homopolymer and copolymer waxes of ethylene and/or propylene can likewise advantageously be used in micronized form in peels and toothpastes as gentle abrasive component.

Using the homopolymer and copolymer waxes produced with metallocene catalysts it is also possible to prepare very solid pastes and sticks with very good solvent binding, in particular of aliphatic solvents.

In a preferred embodiment, the cosmetic, pharmaceutical or dermatological preparations according to the invention comprise one or more homopolymer waxes of the monomer ethylene, wherein the homopolymer waxes have a weight-average molecular weight Mw of less than or equal to 25 000 g/mol, preferably of 1000 to 22 000 g/mol and particularly preferably of 4000 to 20 000 g/mol, a number-average molecular weight Mn of less than or equal to 15 000 g/mol, preferably of 500 to 12 000 g/mol and particularly preferably of 1000 to 5000 g/mol and a molar mass distribution Mw/Mn in the range from 1.5 to 10, preferably from 1.5 to 5, particularly preferably from 1.5 to 3 and especially preferably from 2 to 2.5, and have been obtained by metallocene catalysis.

In a further preferred embodiment, the cosmetic, pharmaceutical or dermatological preparations according to the invention comprise one or more copolymer waxes of the monomers ethylene and propylene, wherein the copolymer waxes have a weight-average molecular weight Mw of less than or equal to 25 000 g/mol, preferably of 2000 to 22 000 g/mol and particularly preferably of 4000 to 20 000 g/mol, a number-average molecular weight Mn of less than or equal to 15 000 g/mol, preferably of 1000 to 12 000 g/mol and particularly preferably of 2000 to 10 000 g/mol, a molar mass distribution Mw/Mn in the range from 1.5 to 10, preferably from 1.5 to 5, particularly preferably from 1.5 to 3 and especially preferably from 2 to 2.5, and have been obtained by metallocene catalysis, and where the copolymer waxes, based on the total weight of the copolymer waxes, comprise 70.0 to 99.9% by weight of structural units originating from the monomer ethylene and 0.1 to 30.0% by weight of structural units originating from the monomer propylene.

In a further preferred embodiment, the cosmetic, pharmaceutical or dermatological preparations according to the invention comprise one or more homopolymer waxes of the monomer propylene, wherein the homopolymer waxes have a weight-average molecular weight Mw of less than or equal to 25 000 g/mol, preferably of 2000 to 22 000 g/mol and particularly preferably of 4000 to 22 000 g/mol, a number-average molecular weight Mn of less than or equal to 15 000 g/mol, preferably of 1000 to 12 000 g/mol and particularly preferably of 2000 to 10 000 g/mol, a molar mass distribution Mw/Mn in the range from 1.5 to 10, preferably from 1.5 to 5, particularly preferably from 1.5 to 3 and especially preferably from 2 to 2.5 and have an isotactic index of greater than 70%, preferably of 75 to 95% and particularly preferably of 80 to 90% and have been obtained by metallocene catalysis.

In a further preferred embodiment, the cosmetic, pharmaceutical or dermatological preparations according to the invention comprise one or more copolymer waxes of the monomers ethylene and propylene, wherein the copolymer waxes have a weight-average molecular weight Mw of less than or equal to 25 000 g/mol, preferably of 2000 to 22 000 g/mol and particularly preferably of 4000 to 20 000 g/mol, a number-average molecular weight Mn of less than or equal to 15 000 g/mol, preferably of 1000 to 12 000 g/mol and particularly preferably of 2000 to 10 000 g/mol, a molar mass distribution Mw/Mn in the range from 1.5 to 10, preferably from 1.5 to 5, particularly preferably from 1.5 to 3 and especially preferably from 2 to 2.5, and have been obtained by metallocene catalysis, and where the copolymer waxes, based on the total weight of the copolymer waxes, comprise 0.1 to 30.0% by weight of structural units originating from the monomer ethylene and 70.0 to 99.9% by weight of structural units originating from the monomer propylene.

In a further preferred embodiment, the cosmetic, pharmaceutical or dermatological preparations according to the invention comprise one or more of the abovedescribed homopolymer and/or copolymer waxes of the monomers ethylene and/or propylene, wherein the homopolymer and copolymer waxes have a ring/sphere dropping or softening point between 80 and 165° C. and a melt viscosity, measured at a temperature of 170° C., between 20 and 40 000 mPa s.

In a further preferred embodiment, the cosmetic, pharmaceutical or dermatological preparations according to the invention comprise one or more of the abovedescribed copolymer waxes of the monomers ethylene and propylene, wherein the latter have a glass transition temperature of at most −20° C.

In a further preferred embodiment, the cosmetic, pharmaceutical or dermatological preparations according to the invention comprise one or more of the abovedescribed homopolymer and/or copolymer waxes of the monomers ethylene and/or propylene, wherein the latter have a degree of crystallization of more than 55% and preferably between 60 and 90%.

In a further preferred embodiment, the cosmetic, pharmaceutical or dermatological preparations according to the invention comprise one or more of the abovedescribed homopolymer and/or copolymer waxes of the monomers ethylene and/or propylene, wherein the latter have a degree of crystallization below 55% and preferably below 50%.

The homopolymer and copolymer waxes of the monomers ethylene and/or propylene used according to the invention in the cosmetic, pharmaceutical or dermatological preparations can be produced, for example, by the process described in EP 571 882 with the metallocene catalysts specified therein. Suitable processes are, for example, also the suspension polymerization, solution polymerization or gas-phase polymerization of the olefins in the presence of metallocene catalysts, with polymerization in the monomers also being possible.

For the purposes of the present invention, homopolymer and/or copolymer waxes produced using metallocene catalysis are understood as meaning either unmodified or polar modified homopolymer and/or copolymer waxes. In the cosmetic, pharmaceutical or dermatological preparations according to the invention, the one or more homopolymer and/or copolymer waxes can either be chosen exclusively from unmodified homopolymer and/or copolymer waxes or exclusively from polar modified homopolymer and/or copolymer waxes, or from mixtures of non-polar and polar modified homopolymer and/or copolymer waxes.

Polar modified waxes can be produced in a known manner from unmodified waxes by oxidation with oxygen-containing gases, for example air, or by graft reaction with polar monomers, for example maleic acid or acrylic acid or derivatives of these acids. The polar modification of metallocene polyolefin waxes by oxidation with air is described, for example, in EP 0 890 583 A1, and the modification by grafting is described, for example, in U.S. Pat. No. 5,998,547.

In a preferred embodiment, the cosmetic, pharmaceutical or dermatological preparations according to the invention comprise one or more homopolymer and/or copolymer waxes produced by means of metallocene catalysis which are not polar modified, and additionally one or more homopolymer and/or copolymer waxes produced by means of metallocene catalysis which are polar modified.

In a further preferred embodiment, the one or more homopolymer and/or copolymer waxes produced by means of metallocene catalysis present in the cosmetic, pharmaceutical or dermatological preparations according to the invention are chosen from non-polar modified homopolymer and/or copolymer waxes.

In a further preferred embodiment, the one or more homopolymer and/or copolymer waxes produced by means of metallocene catalysis present in the cosmetic, pharmaceutical or dermatological preparations according to the invention are chosen from polar modified homopolymer and/or copolymer waxes.

Polar modified homopolymer and/or copolymer waxes are preferably used, for example, when the cosmetic, pharmaceutical or dermatological preparations according to the invention are emulsions.

The profile of properties of the homopolymer and copolymer waxes used according to the invention is determined by the choice of monomers, by their weight ratios, by the average molecular weights Mw and Mn, by the molar mass distribution Mw/Mn, by the tacticities in the case of the propylene homopolymer waxes and by the degree of crystallization of the homopolymer or copolymer waxes.

The melts are clear and transparent. The ring/sphere dropping or softening points are in the range from 80 to 165° C. and preferably in the range from 85 to 145° C. and the densities are in the range from 0.80 to 1.00 g/cm³ and preferably in the range from 0.88 to 0.98 g/cm³. The viscosities for ethylene homopolymer or copolymer waxes, measured at 170° C., are preferably in the range from 20 to 10 000 mPa s and the viscosities for propylene homopolymer or copolymer waxes, measured at 170° C., are preferably in the range from 50 to 10 000 mPa s and particularly preferably in the range from 60 to 6000 mPa s.

The needle penetration values of the homopolymer and copolymer waxes are in the range from less than 1 to 10×0.1 mm and preferably from 1 to 5×0.1 mm.

Upon applying formulations comprising these homopolymer and/or copolymer waxes to the skin or to the hair, water-repellent films form which prevent the migration and the washing out and off of ingredients of the cosmetic, pharmaceutical and dermatological compositions, in particular of active substances and/or pigments.

In addition, the homopolymer and copolymer waxes used according to the invention exhibit a very good solvent-binding capacity, in particular of aliphatic solvents, for example of benzines, white oils and paraffin oil and can be processed with the liquid lipid phase to give solid, thermally stable compositions, for example lipsticks.

Using the abovedescribed odor-neutral white homopolymer and copolymer waxes, it is possible, together with solvents and oil components, to prepare spreadable or flowable creams, cream foams or pastes which have a viscosity at 20° C. of more than 500 mPa s. They are excellent consistency-imparting agents, in particular for formulations based on oil and have good absorption properties which can be utilized for the absorption of oils and the dispersion of pigments, odor substances or solid active ingredients and other solid additives.

The homopolymer and copolymer waxes can very advantageously be used as abrasive component, preferably in dental care compositions and peels.

On account of their hardness, the homopolymer and/or copolymer waxes used according to the invention impart stability to, for example, lipsticks, kohl and mascara sticks, even at elevated temperatures. As binder, the wax can be used together with lanolin, paraffin oil, isopropyl stearate, pigments and perfume for producing eyeshadows, eyebrow pencils, powder and blusher compacts. Here, the water-repellent properties, and also the thickening effect of these homopolymer and copolymer waxes are utilized in order to suppress running of the greasy make-ups.

The abovedescribed homopolymer and/or copolymer waxes are suitable for producing cosmetic, dermatological and pharmaceutical preparations, particularly advantageously for producing decorative cosmetic compositions, sunscreen compositions, deodorants, haircare and styling compositions, cleaning compositions for the skin, in particular peels, and also hair removal compositions.

The preparations according to the invention may be extremely diverse cosmetic, pharmaceutical and dermatological formulations. In particular, they may be pencils, cover sticks, acne sticks, lipsticks, make-ups, foundations, face powder, blusher, mascara, eyeshadows, eyeliners, peel creams, hair waxes, hair styling compositions, styling fluids, hair foams, hair gels, hair sprays, mousse, hair oil, end fluids, hair treatments, night creams, care creams, nutrient creams, perfume creams, body lotions, ointments, lipcare compositions, sunscreen compositions, deodorants, antiperspirants, colored gels in the form of pencils, such as, for example, multiphase pencils, sticks, pastes, powder, creams, cream foams, lotions, self-foaming, foam-like, after-foaming or foamable emulsions, gels, roll-on preparations, foams or depilatories.

In a preferred embodiment, the preparations according to the invention are in the form of sticks or pencils.

In a further preferred embodiment, the preparations according to the invention are in the form of an emulsion.

In a particularly preferred embodiment, the abovedescribed homopolymer and/or copolymer waxes are incorporated in micronized form into emulsions.

The emulsions may either be water-in-oil emulsions or oil-in-water emulsions, microemulsions, nanoemulsions and multiple emulsions. The emulsions can be prepared in a known manner, i.e., for example, by cold, hot, hot/cold or PIT emulsification. A particularly preferred embodiment covers self-foaming, foam-like, after-foaming or foamable emulsions and microemulsions. The preparations according to the invention are characterized by a particularly good adhesion of the cosmetic compositions to the skin and form hydrophobic films which are barely dissolved by the sebum which forms on the skin, meaning that undesired color shifts of the pigments or migration of the active ingredients or solid additives do not occur.

With the help of emulsifiers it is possible to produce a large number of wax preparations. The choice of emulsifier permits the production of nonionogenic and ionogenic wax dispersions.

The emulsions according to the invention comprise at least
a) one of the abovedescribed homopolymer and/or copolymer waxes,
b) an oil component
c) an emulsifier
d) optionally further waxes.

The oil component can advantageously be chosen from the groups of mineral oils, mineral waxes, oils, such as triglycerides, fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number with fatty acids or alkyl benzoates.

A class of oils and fats preferred according to the invention are the triglycerides of linear or branched, saturated or unsaturated, optionally hydroxylated, C₈-C₃₀-fatty acids, in particular vegetable oils, such as sunflower oil, corn oil, soya oil, rice oil, jojoba oil, babassu oil, pumpkin oil, grapeseed oil, sesame oil, walnut oil, apricot oil, orange oil, wheatgerm oil, peach kernel oil, macadamia oil, avocado oil, sweet almond oil, lady's smock oil, castor oil, olive oil, peanut oil, rapeseed oil and coconut oil, and also synthetic triglyceride oils, e.g. the commercial product Myritol®318. Hydrogenated triglycerides are also preferred according to the invention. Oils of animal origin, for example bovine tallow, perhydrosqualene or lanolin, can also be used.

A further class of oils and fats preferred according to the invention are the benzoic acid esters of linear or branched C₈-C₂₂-alkanols, e.g. the commercial products Finsolv®SB (isostearyl benzoate), Finsolv®TN (C₁₂-C₁₅-alkyl benzoate) and Finsolv®EB (ethylhexyl benzoate).

A further class of oils and fats preferred according to the invention are the dialkyl ethers having a total of 8 to 36 carbon atoms, in particular having 12 to 24 carbon atoms, such as, for example, di-n-octyl ether (Cetiol® OE), di-n-decyl ether, di-n-nonyl ether, di-n-undecylether, di-n-dodecyl ether, di-3-ethyldecyl ether, tert-butyl n-octyl ether, isopentyl n-octyl ether, 2-methylpentyl n-octyl ether, n-hexyl n-octyl ether, n-octyl n-decyl ether, n-decyl n-undecyl ether, n-undecyl n-dodecyl ether and n-hexyl n-undecyl ether, and di-tert-butyl ether or diisopentyl ether.

A further class of oils and fats preferred according to the invention are hydrocarbon oils, for example those having linear or branched, saturated or unsaturated C₇-C₄₀-carbon chains, for example petroleum jelly, dodecane, isododecane, cholesterol, lanolin, hydrogenated polyisobutylene, docosane, hexadecane, isohexadecane, paraffin oils, paraffin waxes, isoparaffin oils, e.g. the commercial products of the Permethyl® series, squalane, squalene, synthetic hydrocarbons such as polyisobutene and alicyclic hydrocarbons, e.g. the commercial product 1,3-di(2-ethylhexyl)cyclohexane (Cetiol®S), ozokerite, microwaxes and ceresin.

Likewise suitable are branched saturated or unsaturated fatty alcohols having 6-30 carbon atoms, e.g. isostearyl alcohol, and Guerbet alcohols.

A further class of oils and fats preferred oils according to the invention are alkyl hydroxy-carboxylates. Preferred alkyl hydroxycarboxylates are full esters of glycolic acid, lactic acid, malic acid, tartaric acid or citric acid. Further hydroxycarboxylic acids which are suitable in principle are esters of β-hydroxypropionic acid, of tartronic acid, of D-gluconic acid, saccharic acid, mucic acid or glucuronic acid. Suitable alcohol components of these esters are primary, linear or branched aliphatic alcohols having 8 to 22 carbon atoms. In this case, esters of C₁₂-C₁₅-fatty alcohols are particularly preferred. Esters of this type are commercially obtainable, e.g. under the trade name Cosmacol® of EniChem, Augusta

Industriale.A further class of oils and fats preferred according to the invention are dicarboxylic acid esters of linear or branched C₂-C₁₀-alkanols, such as di-n-butyl adipate (Cetiol®B), di(2-ethylhexyl) adipate and di(2-ethylhexyl) succinate, and diol esters such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate.

Likewise preferred oils and fats are symmetrical, asymmetrical or cyclic esters of carbonic acid with fatty alcohols, glycerol carbonate or dicaprylyl carbonate (Cetiol®CC).

A further class of oils and fats preferred according to the invention are the esters of dimers of unsaturated C₁₂-C₂₂-fatty acids (dimer fatty acids) with monohydric linear, branched or cyclic C₂-C₁₈-alkanols or with polyhydric linear or branched C₂-C₆-alkanols.

In a further preferred embodiment of the invention, if the oil component is a silicone oil, the preparations according to the invention are preferably present in the form of a water-in-silicone emulsion and comprise water, silicone, one or more emulsifiers and one or more copolymer waxes.

Silicone oils and waxes which are preferably available are dimethylpolysiloxanes and cyclomethicones, polydialkylsiloxanes R₃SiO(R₂SiO)ₓSiR₃, where R is methyl or ethyl, particularly preferably methyl, and x is a number from 2 to 500, for example the dimethicones obtainable under the trade names VICASIL (General Electric Company), DOW CORNING 200, DOW CORNING 225, DOW CORNING 200 (Dow Corning Corporation), and the dimethicones obtainable under SilCare® Silicone 41M65, SilCare® Silicone 41M70, SilCare® Silicone 41M80 (Clariant GmbH), stearyldimethylpolysiloxane, C₂₀-C₂₄-alkyldimethylpolysiloxane, C₂₄-C₂₈-alkyl-dimethylpolysiloxane, but also the methicones obtainable under SilCare® Silicone 41M40, SilCare® Silicone 41M50 (Clariant GmbH), furthermore trimethylsiloxy-silicates [(CH₂)₃SiO)_{1/2}]ₓ[SiO₂]_{y}, where x is a number from 1 to 500 and y is a number from 1 to 500, dimethiconols R₃SiO[R₂SiO]ₓSiR₂OH and HOR₂SiO[R₂SiO]ₓSiR₂OH, where R is methyl or ethyl and x is a number up to 500, polyalkylarylsiloxanes, for example the polymethylphenylsiloxanes obtainable under the trade names SF 1075 METHYLPHENYL FLUID (General Electric Company) and 556 COSMETIC GRADE PHENYL TRIMETHICONE FLUID (Dow Corning Corporation), polydiarylsiloxanes, silicone resins, cyclic silicones and amino-, fatty acid-, alcohol-, polyether-, epoxy-, fluoro- and/or alkyl-modified silicone compounds, and also polyethersiloxane copolymers.

Preparations present according to the invention as emulsions comprise one or more emulsifiers from the group consisting of the nonionic, anionic, cationic or amphoteric emulsifiers.

Nonionic emulsifiers which are available are addition products of 0 to 30 mol of ethylene oxide and/or 0 to 5 mol of propylene oxide to linear fatty alcohols having 6 to 30 carbon atoms, preferably 10 to 22 carbon atoms, and very particularly preferably 14 to 22 carbon atoms. Those which can be used are, for example, octanol (capryl alcohol), octenol, octadienol, decanol (capric alcohol), decenol, decadienol, dodecanol (lauryl alcohol), dodecadienol, ricinoleyl alcohol, myristyl alcohol, cetyl alcohol, stearyl alcohol, cetearyl alcohol, oleyl alcohol, linoleyl alcohol, linolenyl alcohol, arachidyl alcohol, behenyl alcohol. Those which can also be used are fatty alcohol cuts which are obtained by reduction of naturally occurring triglycerides, such as bovine tallow, palm oil, peanut oil, rapeseed oil, cottonseed oil, soya oil, sunflower oil and linseed oil or which are produced from transesterification products with corresponding alcohols from fatty acid esters and thus are a mixture of different fatty alcohols. Such substances are commercially obtainable, for example, under the name Stenol®, e.g. Stenol® 1618, or Lanette®, e.g. Lanette® O and Lanette®22, or Lorol®, e.g. Lorol®C18.

A further class of emulsifiers preferred according to the invention are addition products of 0 to 30 mol of ethylene oxide and/or 0 to 5 mol of propylene oxide onto linear and/or branched, saturated and/or unsaturated fatty acids having 6 to 30 carbon atoms, preferably 10 to 22 carbon atoms. Isostearic acid, such as the commercial products Emersol®871 and Emersol®875, isopalmitic acids such as Edenor®IP95, and all further fatty acids commercially available under the trade name Edenor® (Cognis) may be mentioned. Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeosteric acid, arachic acid, gadoleic acid, behenic acid, erucic acid and dimers of unsaturated fatty acids, and their technical mixtures. Fatty acid cuts from coconut oil or palm oil are particularly preferred. Stearic acid is especially preferred.

Customarily, the fatty acids are neutralized using a basic agent, e.g. NaOH, and used, for example, in the form of their sodium, potassium, ammonium, calcium, magnesium and zinc salts.

A further class of emulsifiers which can be used are esters of, if desired, alkylated sugars with C₆-C₃₀-fatty acids. Sugars which can be used are any desired mono- or oligosaccharides. Customarily, monosaccharides having 5 or 6 carbon atoms are used, for example ribose, xylose, lyxose, altose, glucose, fructose, galactose, arabinose, altrose, mannose, gulose, idose, talose, and the deoxy sugars rhamnose and fucose. Sugars having 4 carbon atoms can also be used, e.g. erythrose and threose. Oligosaccharides suitable according to the invention are composed of two to 10 monosaccharide units, e.g. sucrose (saccharose), lactose or trehalose. Preferred sugar units are the monosaccharides glucose, fructose, galactose, arabinose and the disaccharide sucrose. Glucose and sucrose are particularly preferred. The sugars can be partially etherified with methyl, ethyl, propyl, isopropyl or butyl groups, e.g. methyl glucoside, ethyl glucoside or butyl glucoside. For esterification, all C₆-C₃₀-fatty acids and their mixtures can be used which have been mentioned above. In principle, mono- and polyesterified sugars are suitable. The mono-, sesqui- and diesters are preferred, for example sucrose monostearate, sucrose distearate, sucrose monococoate, sucrose dicocoate, methyl glucoside monostearate, methyl glucoside sesquistearate, methyl glucoside isostearate, ethyl glucoside monolaurate, ethyl glucoside dilaurate, ethyl glucoside monococoate, ethyl glucoside dicocoate and butyl glucoside monococoate.

A further class of suitable emulsifiers are C₈-C₂₂-alkyl mono- and oligoglycosides, corresponding to the formula RO-(Z)ₓ, where R is a C₈-C₂₂-alkyl group, Z is a sugar and x is the number of sugar units. The alkyl mono- and oligoglycosides which can be used according to the invention can only contain a certain alkyl radical R. Particularly preferred alkylmono- and oligoglycosides are those in which R essentially comprises C₈-and C₁₀-alkyl groups, essentially C₁₂- and C₁₄-alkyl groups, essentially C₈- to C₁₆-alkyl groups or essentially C₁₂- to C₁₆-alkyl groups. The sugar unit Z used can be any desired mono- or oligosaccharide, such as have been mentioned above. Preferred sugar units are glucose, fructose, galactose, arabinose and sucrose, glucose being particularly preferred. The alkyl mono- and oligoglycosides which can be used according to the invention on average contain 1.1-5, preferably 1.1-2.0 and particularly preferably 1.1-1.8, sugar units.

The alkoxylated homologs of the alkyl mono- and oligoglycosides mentioned can also be used according to the invention. Suitable are, for example, cocoyl glucoside, decyl glucoside, lauryl glucoside, cetearyl glucoside and arachidyl glucoside.

Besides the alkyl mono- and oligoglucosides mentioned, the mixtures of alkyl mono- and oligoglucosides and fatty alcohols, e.g. the commercially obtainable products Montanov®68 and Montanov®202, are particularly preferred.

A further class of preferred emulsifiers are the partial esters of propylene glycol, glycerol and sorbitan with C₈-C₂₂-fatty acids. For esterification, all C₈-C₂₂-fatty acids and their mixtures can be used which have already been mentioned above. Particularly suitable examples are propylene glycol monostearate, glycerol monolaurate, glycerol monostearate, glycerol distearate, glycerol monooleate, sorbitan monolaurate, sorbitan dilaurate, sorbitan monostearate, sorbitan sesquistearate, sorbitan distearate, sorbitan monoisostearate, sorbitan monooleate, sorbitan dioleate or the commercial products Monomuls®90-0, Monomuls®90-L 12 and Cutina®MD. These emulsifiers can on average contain up to 10 ethylene oxide and/or propylene oxide units per molecule.

A further preferred class of emulsifiers are polyglycerols of the formula HO—CH₂—CHOH—CH₂[—O—CH₂—CHOH—CH₂]ₙ—O—CH₂—CHOH—CH₂OH with n=0-8 and their esters with linear and branched C₈-C₂₂-fatty acids, which can carry functional groups in the alkyl chain, preferably polyglyceryl 2-dipolyhydroxystearate (commercial product Dehymuls® PGPH) and polyglyceryl 3-diisostearate (commercial product Lameform® TGI).

A further class of preferred emulsifiers are sterols, in particular cholesterol, lanosterol, β-sitosterol, stigmasterol, campesterol and ergosterol, and mycosterols. Customary commercial sterol emulsifiers are produced on the basis of soya or rapeseed sterols. According to the invention, the employment of sterols which contain 5-10 ethylene oxide units per molecule is preferred. For example, the commercial products Generol®122, Generol® 122 E 5, Generol® 122 E 10 and Generol®RE-10 are suitable.

Emulsifiers which can likewise be used with preference are phospholipids, especially the phosphatidylcholines or lecithins. Phospholipids are phosphoric acid diesters, more rarely monoesters, of mostly linear saturated and unsaturated C₈-C₂₂-fatty acids. Soya lecithin is preferred.

A further class of preferred emulsifiers are the esterification products of lactic acid or glycolic acid with linear or branched C₈-C₂₂-fatty acids, and the sodium, potassium, ammonium, calcium, magnesium and zinc salts of these esterification products.

Particular preference is given to esterification products of the formula (5) 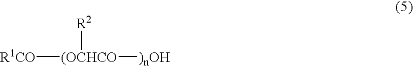
where R¹ is a linear or branched saturated or unsaturated alkyl radical having 5 to 21 carbon atoms and R² is a methyl group or a hydrogen atom and n is an integer from 1-4.

Among the acyl radicals R¹CO—, the radicals selected from the caproyl, capryloyl, caprinoyl, lauroyl, myristoyl, cetoyl, palmitoyl, stearoyl, isostearoyl and the oleyl group are in turn preferred. The stearoyl and the isostearoyl group are particularly preferred.

The radical R² is preferably methyl.

The degree of oligomerization n is preferably 1 or 2.

The compound sodium stearoyl-2-lactylate is especially preferred.

A further class of preferably used emulsifiers are phosphoric acid mono-, di- and triesters of saturated or unsaturated linear or branched fatty alcohols having 8 to 30 carbon atoms and their ethylene oxide adducts having 1-10 ethylene oxide groups per molecule. These alkyl and alkenyl phosphates are shown in the formula (6) 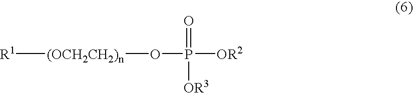
in which R¹ is a saturated or unsaturated, linear or branched hydrocarbon radical having 8 to 30 carbon atoms, R² and R³ independently of one another are a hydrogen atom, X or a radical (CH₂CH₂O)ₙR¹, n is numbers from 0 to 10 and X is an alkali metal or alkaline earth metal cation or a cation NR⁴R⁵R⁶R⁷, with R⁴ to R⁷ independently of one another being a C₁-C₄-hydrocarbon radical.

The alkyl and alkenyl phosphates preferred according to the invention contain, as a group R¹, alkyl radicals having 12-18 carbon atoms, which can be saturated or unsaturated and linear or branched. These groups R¹ are in particular lauryl, myristyl, cetyl, palmityl, stearyl, isostearyl and oleyl. Preferred values for n are either 0 or values from 1-10, preferably 2-5, particularly preferably 3-4 (alkyl or alkenyl ether phosphates). Furthermore, the use of ester mixtures of mono-, di- and tri-esters is preferred, where the proportion of mono- and diesters predominates compared with the proportion of triester. The use of pure triesters can, however, also be preferred. Suitable commercial products are derived, for example, from the Hostaphat® series (Clariant), e.g. Hostaphat®KW 340 D, Hostaphat®KO300 N, Hostaphat®KO380 and Hostaphat®KL 340.

A further class of emulsifiers preferably used according to the invention are acyl glutamates of the formula (7) 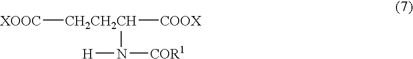
in which R¹CO is a linear or branched acyl radical having 6 to 22 carbon atoms and 0, 1, 2 or 3 double bonds and X is hydrogen, an alkali metal or alkaline earth metal cation, an ammonium, alkylammonium, alkanolammonium and/or glucammonium, for example acylglutamates which are derived from fatty acids having 6 to 22, preferably 12 to 18 carbon atoms, such as, for example, C_{12/14}-and/or C_{12/18}-coconut fatty acid, lauric acid, myristic acid, palmitic acid and/or stearic acid, in particular sodium N-cocoyl- and sodium N-stearoyl-L-glutamate.

A further class of emulsifiers preferred according to the invention are the esters of a hydroxy-substituted di- or tricarboxylic acid of the formula (8), 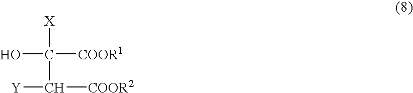
in which X is H or a —CH₂COOR group, Y is H or —OH, with the condition that Y is H if X is —CH₂COOR, R, R¹ and R² independently of one another are a hydrogen atom, an alkali metal or an alkaline earth metal cation, an ammonium group, the cation of an organoammonium base or a radical Z which is derived from a polyhydroxylated organic compound which is selected from the group consisting of the etherified (C₆-C₁₈)-alkylsaccharides having 1 to 6 monomeric saccharide units and/or the etherified aliphatic (C₆-C₁₆)-hydroxyalkylpolyols having 2 to 16 hydroxyl radicals, with the proviso that at least one of the groups R, R¹ or R² is a radical Z.

A further class of emulsifiers preferred according to the invention are the esters of the sulfosuccinic acid salt of the formula (9) 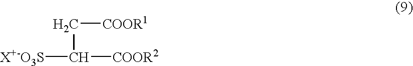
in which R¹ and R² independently of one another are a hydrogen atom, an alkali metal or alkaline earth metal cation, an ammonium group, the cation of an organo-ammonium base or a radical Z which is derived from a polyhydroxylated organic compound which is selected from the group consisting of the etherifed (C₆-C₁₈)-alkylpolysaccharides having 1 to 6 monomeric saccharide units and/or the etherified aliphatic (C₆-C₁₈)-hydroxyalkylpolyols having 2 to 16 hydroxyl radicals, with the proviso that at least one of the groups R¹ or R² is a radical Z, and X⁺ is an alkali metal or alkaline earth metal cation, an ammonium group or the cation of an organo-ammonium base.

A further class of emulsifiers preferred according to the invention are the sulfosuccinic acid mono- and dialkyl esters having 8 to 24 carbon atoms in the alkyl group and sulfosuccinic acid monoalkylpolyoxyesters having 8 to 24 carbon atoms in the alkyl group and 1 to 6 ethoxy groups, and their alkali metal, alkaline earth metal or ammonium salts.

A further class of emulsifiers preferred according to the invention are the esters of tartaric acid and citric acid with alcohols, which are addition products of approximately 2 to 10 molecules of ethylene oxide and/or propylene oxide onto fatty alcohols having 8 to 22 carbon atoms, and their alkali metal, alkaline earth metal or ammonium salts.

Further emulsifiers preferred according to the invention are ether carboxylic acids of the formula R—O—(CH₂—CH₂O)ₓ—CH₂—COOH, in which R is a linear alkyl group having 8 to 30 carbon atoms and x is 0 or 1 to 10, acyl sarcosinates having a linear or branched acyl radical having 6 to 22 carbon atoms and 0, 1, 2 or 3 double bonds, acyl taurates having a linear or branched acyl radical having 6 to 22 carbon atoms and 0, 1, 2 or 3 double bonds, and acyl isethionates having a linear or branched acyl radical having 6 to 22 carbon atoms and 0, 1, 2 or 3 double bonds, and the alkali metal, alkaline earth metal or ammonium salts of these emulsifiers.

Amphoteric emulsifiers which are preferably available are alkylaminoalkylcarboxylic acids, betaines, sulfobetaines and imidazoline derivatives.

Preferably, fatty alcohol ethoxylates are chosen from the group consisting of the ethoxylated stearyl alcohols, cetyl alcohols, cetylstearyl alcohols, in particular polyethylene glycol(13) stearyl ether, polyethylene glycol(14) stearyl ether, polyethylene glycol(15) stearyl ether, polyethylene glycol(16) stearyl ether, polyethylene glycol(17) stearyl ether, polyethylene glycol(18) stearyl ether, polyethylene glycol(19) stearyl ether, polyethylene glycol(20) stearyl ether, polyethylene glycol(12) isostearyl ether, polyethylene glycol(13) isostearyl ether, polyethylene glycol(114) isostearyl ether, polyethylene glycol(15) isostearyl ether, polyethylene glycol(16) isostearyl ether, polyethylene glycol(17) isostearyl ether, polyethylene glycol(18) isostearyl ether, polyethylene glycol(19) isostearyl ether, polyethylene glycol(20) isostearyl ether, polyethylene glycol(13) cetyl ether, polyethylene glycol(14) cetyl ether, polyethylene glycol(15) cetyl ether, polyethylene glycol(16) cetyl ether, polyethylene glycol(17) cetyl ether, polyethylene glycol(18) cetyl ether, polyethylene glycol(19) cetyl ether, polyethylene glycol(20) cetyl ether, polyethylene glycol(13) isocetyl ether, polyethylene glycol(14) isocetyl ether, polyethylene glycol(15) isocetyl ether, polyethylene glycol(16) isocetyl ether, polyethylene glycol(17) isocetyl ether, polyethylene glycol(18) isocetyl ether, polyethylene glycol(19) isocetyl ether, polyethylene glycol(20) isocetyl ether, polyethylene glycol(12) oleyl ether, polyethylene glycol(13) oleyl ether, polyethylene glycol(14) oleyl ether, polyethylene glycol(15) oleyl ether, polyethylene glycol(12) lauryl ether, polyethylene glycol(12) isolauryl ether, polyethylene glycol(13) cetylstearyl ether, polyethylene glycol(14) cetylstearyl ether, polyethylene glycol(15) cetylstearyl ether, polyethylene glycol(16) cetylstearyl ether, polyethylene glycol(17) cetylstearyl ether, polyethylene glycol(18) cetylstearyl ether, polyethylene glycol(19) cetylstearyl ether, polyethylene glycol(20) cetylstearyl ether.

Preferably, fatty acid ethoxylates are chosen from the group consisting of the ethoxylated stearates, isostearates and oleates, in particular polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(117) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(116) oleate, polyethylene glycol(17) oleate, polyethylene glycol(18) oleate, polyethylene glycol(19) oleate, polyethylene glycol(20) oleate.

The ethoxylated alkyl ether carboxylic acid or its salts used can advantageously be sodium laureth(11EO) carboxylate.

An advantageous alkyl ether sulfate is lauryl diglycol ether sulfate sodium salt, an advantageous ethoxylated cholesterol derivative is polyethylene glycol(30) cholesteryl ether. Polyethylene glycol(25) soya sterol is likewise preferred.

The ethoxylated triglycerides used can advantageously be polyethylene glycol(60) evening primose glycerides.

It is furthermore advantageous to choose the polyethylene glycol glycerol fatty acid esters from the group consisting of polyethylene glycol(20) glyceryl laurate, polyethylene glycol(6) glyceryl caproate/caprate, polyethylene glycol(20) glyceryl oleate, polyethylene glycol(20) glyceryl isostearate and polyethylene glycol(18) glyceryl oleate/cocoate.

Among the sorbitan esters, polyethylene glycol (20)sorbitan monolaurate, polyethylene glycol (20)sorbitan monostearate, polyethylene glycol (20)sorbitan monoisostearate, polyethylene glycol (20)sorbitan monopalmitate and polyethylene glycol (20)sorbitan monooleate are particularly suitable.

Advantageous W/O emulsifiers which can be used are: fatty alcohols having 8 to 30 carbon atoms, monoglycerol esters of saturated and/or unsaturated, branched and/or unbranched alkanecarboxylic acids of a chain length of from 8 to 24, in particular 12 to 18, C atoms, diglycerol esters of saturated and/or unsaturated, branched and/or unbranched alkanecarboxylic acids of a chain length of from 8 to 24, in particular 12 to 18, carbon atoms, monoglycerol ethers of saturated and/or unsaturated, branched and/or unbranched alcohols of a chain length of from 8 to 24, in particular 12 to 18, carbon atoms, diglycerol ethers of saturated and/or unsaturated, branched and/or unbranched alcohols of a chain length of from 8 to 24, in particular 12 to 18, carbon atoms, propylene glycol esters of saturated and/or unsaturated, branched and/or unbranched alkanecarboxylic acids of a chain length of from 8 to 24, in particular 12 to 18, carbon atoms, and sorbitan esters of saturated and/or unsaturated, branched and/or unbranched alkanecarboxylic acids of a chain length of from 8 to 24, in particular 12 to 18, carbon atoms.

Especially advantageous W/O emulsifiers are glyceryl monostearate, glyceryl monoisostearate, glyceryl monomyristate, glyceryl monooleate, glyceryl monolaurate, glyceryl monocaprylate, glyceryl monocaprinate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, saccharose distearate, cetyl alcohol, stearyl alcohol, arachidyl alcohol, behenyl alcohol, isobehenyl alcohol, selachyl alcohol, chimyl alcohol or polyethylene glycol(2) stearyl ether.

In the compositions according to the invention, mixtures of compounds from a number of these substance classes can be present.

In a further particularly preferred embodiment, the preparations according to the invention are present in the form of oil-in-water emulsions.

In an especially preferred embodiment, the preparations according to the invention are in the form of emulsions of the oil-in-water type, preferably cosmetic or dermatological emulsions of the oil-in-water type, and comprise, based on the total weight of the preparation,
a) up to 95% by weight, preferably 60 to 92% by weight, particularly preferably 70 to 90% by weight, especially preferably 75 to 85% by weight, of a water phase,
b) up to 40% by weight, preferably 1 to 40% by weight, particularly preferably 2 to 25% by weight, especially preferably 5 to 20% by weight, of an oil phase,
c) up to 15% by weight, preferably 0.5 to 12% by weight, particularly preferably 1 to 8% by weight, especially preferably 1 to 5% by weight, of one or more emulsifiers and
d) up to 5% by weight, preferably 0.01 to 5% by weight, particularly preferably 0.05 to 3% by weight, especially preferably 0.1 to 2% by weight, of homopolymer and/or copolymer wax.

In a further particularly preferred embodiment, the preparations according to the invention are in the form of gel creams of the oil-in-water type, preferably cosmetic or dermatological gel creams of the oil-in-water type, and comprise, based on the total weight of the preparation,
a) up to 95% by weight, preferably 50 to 95% by weight, particularly preferably 70 to 90% by weight, especially preferably 75 to 85% by weight, of a water phase,
b) up to 30% by weight, preferably 1 to 30% by weight, particularly preferably 3 to 25% by weight, especially preferably 5 to 15% by weight, of an oil phase,
c) up to 5% by weight, preferably 0.5 to 5% by weight, particularly preferably 0.2 to 4% by weight, especially preferably 0.5 to 3% by weight, of one or more emulsifiers and
d) up to 5% by weight, preferably 0.01 to 5% by weight, particularly preferably 0.05 to 3% by weight, especially preferably 0.1 to 2% by weight, of homopolymer and/or copolymer wax.

In a further particularly preferred embodiment, the preparations according to the invention are in the form of water-in-oil emulsions.

In an especially preferred embodiment, the preparations according to the invention are in the form of emulsions of the water-in-oil type, preferably cosmetic or dermatological emulsions of the water-in-oil type, and comprise, based on the total weight of the preparations,
a) up to 95% by weight, preferably 40 to 95% by weight, particularly preferably 50 to 90% by weight, especially preferably 60 to 85% by weight, of a water phase,
b) up to 60% by weight, preferably 2 to 60% by weight, particularly preferably 5 to 40% by weight, especially preferably 10 to 30% by weight, of an oil phase,
c) up to 20% by weight, preferably 0.5 to 20% by weight, particularly preferably 1 to 15% by weight, especially preferably 4 to 12% by weight, of one or more emulsifiers and
d) up to 5% by weight, preferably 0.01 to 5% by weight, particularly preferably 0.05 to 3% by weight, especially preferably 0.1 to 2% by weight, of homopolymer and/or copolymer wax.

In a further especially preferred embodiment, the preparations according to the invention are in the form of water-in-silicone emulsions, preferably in the form of cosmetic or dermatological water-in-silicone emulsions, and comprise, based on the total weight of the preparation,
a) up to 90% by weight, preferably 20 to 90% by weight, particularly preferably 40 to 85% by weight, especially preferably 60 to 80% by weight, of a water phase,
b) up to 80% by weight, preferably 10 to 70% by weight, particularly preferably 20 to 60% by weight, especially preferably 30 to 50% by weight, of silicone oil,
c) 0.5 to 20% by weight, preferably 1 to 15% by weight, particularly preferably 3 to 10% by weight, of one or more emulsifiers and
d) 0.01 to 5% by weight, preferably 0.05 to 3% by weight, particularly preferably 0.1 to 2% by weight, of homopolymer and/or copolymer wax.

In a further preferred embodiment, the homopolymer and/or copolymer waxes used according to the invention are used in micronized form, preferably in particle sizes of from 4 to 45 μm and especially preferably in particle sizes of from 4 to 20 μm.

Micronized homopolymer and/or copolymer waxes can be obtained, for example, by very fine grinding on suitable jet-mills or mechanical mills or by spraying from the melt. The average particle size (so-called d50 value) of micronized waxes obtained in this way is usually between 3 and 30 μm and preferably between 5 and 15 μm.

The micronized homopolymer and/or copolymer waxes can be readily dispersed, bring about better slide properties of the compositions and improve the feel on the skin and the spreadability of the compositions on the skin and on the hair. The homopolymer and/or copolymer waxes described above can particularly advantageously be incorporated into peels for cleansing and caring for the skin.

In a further preferred embodiment, the preparations according to the invention are in the form of a dispersion and comprise

a) a carrier material, preferably one or more oil components and/or solvents,

b) one or more emulsifiers and

c) one or more of the homopolymer and/or copolymer waxes described above and optionally one or more further waxes.

In a further preferred embodiment, the preparations according to the invention are in the form of wax dispersions, comprising

a) a carrier material, preferably one or more oil components and/or solvents,

b) one or more emulsifiers and

c) one or more of the homopolymer and/or copolymer waxes described above and

d) optionally one or more further waxes

where the wax content is 20 to 45% by weight. The wax dispersions can be incorporated into cosmetic, pharmaceutical and dermatological preparations.

Besides the homopolymer and/or copolymer waxes or micronized homopolymer and/or copolymer waxes, the cosmetic, pharmaceutical and dermatological preparations or wax dispersions according to the invention can comprise further waxes, such as oxidized polyethylene waxes, amide waxes, carnauba waxes, montan waxes, paraffin waxes, Fischer-Tropsch waxes, optionally in combination with hydrophilic waxes, such as, for example, cetylstearyl alcohol.

As further waxes, the cosmetic preparations according to the invention can particularly preferably comprise copolymer waxes comprising

a) one or more structural units —CH₂—CHR—, where R is a linear or branched alkyl group having 24 to 58 carbon atoms,

b) optionally one or more structural units 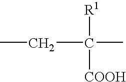
where R¹ is hydrogen or methyl,
   c) one or more structural units 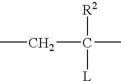
where
R² is hydrogen or methyl,
L is —COOR³, —CONR⁷R⁸ or —COO⁻X⁺,
   R³ is a straight-chain or branched alkyl group having 1 to 36 carbon atoms or a straight-chain or branched alkenyl group having 2 to 36 carbon atoms, which may optionally also be alkoxylated and can preferably comprise ethyleneoxy (EO), propyleneoxy (PO), butyleneoxy (BO) or EO/PO groups, or
is a group (AO)ₙ—H, where (AO) is an ethoxy, propoxy or butoxy group and x is a number from 1 to 50, or
is a glycidyl group, a C₂-C₁₀-hydroxyalkyl group or a glycerol group, or
is a cyclic aromatic or nonaromatic group, preferably a cycloalkyl group, having 5 to 8, preferably 6, ring atoms, or
is a cyclic aromatic or nonaromatic group having 5 to 8, preferably 6, ring atoms, where the ring is formed from carbon atoms and heteroatoms, preferably O and/or N, and both the carbon atoms and the nitrogen atoms may be substituted by linear or branched alkyl or alkoxy groups having 1 to 36 carbon atoms or by linear or branched alkenyl or alkenyloxy groups having 2 to 36 carbon atoms, or by acetyl groups —COR⁴, in which R⁴ is an alkyl group having 1 to 22 carbon atoms, or
   is —CR⁵R⁶)_{y}-cycloalkyl or is —CR⁵R⁶)_{y}-aryl, where R⁵ and R⁶, in each case independently of one another, are H or a linear or branched alkyl group having 1 to 4 carbon atoms, and y is a number from 1 to 10, or is a perfluoroalkyl group having 8 to 18 carbon atoms,

R⁷ and R⁸, in each case independently of one another, are hydrogen, or are a straight-chain or branched alkyl group having 1 to 36 carbon atoms or a straight-chain or branched alkenyl group having 2 to 36 carbon atoms, which may optionally also be alkoxylated and can preferably comprise ethyleneoxy (EO), propyleneoxy (PO), butyleneoxy (BO) or EO/PO groups, or

is a (C₂-C₁₀)-hydroxyalkyl group, or

is —CH₂—CH₂—N(CH₃)₂ or is a polyamine radical, or

is a cyclic aromatic or nonaromatic group, preferably a cycloalkyl group, having 5 to 8, preferably 6, ring atoms, or

is a cyclic aromatic or nonaromatic group having 5 to 8, preferably 6, ring atoms, where the rings are formed from carbon atoms and heteroatoms, preferably O and/or N, and both the carbon atoms and also the nitrogen atoms may be substituted by linear or branched alkyl or alkoxy groups having 1 to 36 carbon atoms or by linear or branched alkenyl or alkenyloxy groups having 2 to 36 carbon atoms, or by acetyl groups —COR⁹, in which R⁹ is an alkyl group having 1 to 22 carbon atoms, or

R⁷ and R⁸, together with the nitrogen atom to which they are bonded, form a 5-, 6- or 7-membered aromatic or nonaromatic ring, and, besides the nitrogen atom, the rings preferably contain only CH₂ groups,

X⁺ is Li⁺, Na⁺, K⁺, Mg⁺⁺/2, Ca⁺⁺/2, Al⁺⁺⁺/3, NH₄⁺, a monoalkylammonium ion, dialkylammonium ion, trialkylammonium ion and/or tetraalkylammonium ion, where the alkyl substituents of the ammonium ions may, independently of one another, be (C₁-C₂₂)-alkyl radicals or (C₂-C₁₀)-hydroxyalkyl radicals, and

d) optionally one or more structural units which are derived from styrene, 3-methylstyrene, 4-methylstyrene or α-methylstyrene.

In a further preferred embodiment, the preparations according to the invention are in the form of decorative compositions.

A further preferred embodiment covers cosmetic and dermatological sticks, for example lipsticks, suncream sticks, antiacne sticks, eyebrow pencils, cover sticks and deodorant sticks, comprising
a) a lipid phase of at least one oil component and at least one homopolymer and/or copolymer wax, as described above,
b) optionally substances which are soluble or dispersible in the lipid phase,
c) an aqueous phase,
d) optionally substances which are soluble or dispersible in water,
e) optionally one or more active substances, and
f) at least one W/O emulsifier,
   where the proportion of the aqueous phase, based on the finished preparation, can be 30 to 80% by weight.

In a further preferred embodiment, especially where decorative compositions are concerned, the cosmetic and dermatological preparations according to the invention comprise one or more colorants, preferably chosen from color lakes, toners and pigments. In this context, they are preferably present in the form of powders, compacts, pastes, creams or sticks.

In a particularly preferred embodiment, the preparations according to the invention are in the form of suspensions and comprise, based on the total weight of the preparations,
a) 0.1 to 10% by weight, preferably 0.2 to 6% by weight, particularly preferably 0.3 to 5% by weight, of homopolymer and/or copolymer wax and
b) 0.1-30% by weight, preferably 0.5 to 15% by weight, particularly preferably 1.0 to 10% by weight, of solid particles, in particular chosen from the group of dyes, color-imparting pigments, effect and photoprotective pigments, adsorbents and abrasive components.

In a further particularly preferred embodiment, the preparations according to the invention are in the form of gel-based eyeshadows and comprise, based on the total weight of the preparations,
a) 0.1 to 10% by weight, preferably 0.2 to 6% by weight, particularly preferably 0.3 to 5% by weight, of homopolymer and/or copolymer wax and
b) 0.1 to 30% by weight, preferably 0.5 to 15% by weight, particularly preferably 1.0 to 10% by weight, of dyes and/or color-imparting pigments.

The preparations according to the invention can comprise solid inorganic and organic particles. For decorative cosmetics, colored and also colorless pigments are used. Some of the pigments specified below also serve as UV absorbers and/or photoprotective pigments.

The dyes and pigments, both organic and inorganic dyes, can be chosen from the appropriate positive list of the Cosmetics Directive and/or the EC list of cosmetic colorants. The following substances can, for example, be used in the preparations according to the invention.

| Chemical or other name | CIN | Color |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2- | 12085 | red |
| hydroxynaphthalene | | |
| Pigment Red 3 | 12120 | red |
| Cerise Red; Sudan Red; Fat Red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfonic acid diethylamide-1′- | 12490 | red |
| phenylazo)-3-hydroxy-5″-chloro-2″,4″-dimethoxy-2- | | |
| naphthoic acid anilide | | |
| Disperse Yellow 16 | 12700 | yellow |
| 1-(4-Sulfo-1-phenylazo)-4-aminobenzenesulfonic | 13015 | yellow |
| acid | | |
| 2,4-Dihydroxyazobenzene-4′-sulfonic acid | 14270 | orange |
| 2-(2,4-Dimethylphenylazo-5-sulfonic acid)-1- | 14700 | red |
| hydroxynaphthalene-4-sulfonic acid | | |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfonic acid-4-chloro-5-carboxylic acid-1- | 15525 | red |
| phenylazo)-2-hydroxynaphthalene | | |
| 1-(3-Methylphenylazo-4-sulfonic acid)-2- | 15580 | red |
| hydroxynaphthalene | | |
| 1-(4′,(8′)-Sulfonic acid naphthylazo)-2- | 15620 | red |
| hydroxynaphthalene | | |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2- | 15850 | red |
| naphthylcarboxylic acid | | |
| 1-(2-Sulfo-4-methyl-5-chloro-1-phenylazo)-2- | 15865 | red |
| hydroxynaphthalene-3-carboxylic acid | | |
| 1-(2-Sulfo-1-naphthylazo)-2-hydroxynaphthalene- | 15880 | red |
| 3-carboxylic acid | | |
| 1-(3-Sulfo-1-phenylazo)-2-naphthol-6-sulfonic acid | 15980 | orange |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6-disulfonic | 16185 | red |
| acid | | |
| Acid Orange 10 | 16230 | orange |
| 1-(4-Sulfo-1-naphthylazo)-2-naphthol-6,8-disulfonic | 16255 | red |
| acid | | |
| 1-(4-Sulfo-1-naphthylazo)-2-naphthol-3,6,8- | 16290 | red |
| trisulfonic acid | | |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5- | 19140 | yellow |
| hydroxypyrazolone-3-carboxylic acid | | |
| Pigment Yellow 16 | 20040 | yellow |
| 2,6-(4′-Sulfo-2″,4″-dimethyl)bisphenylazo)-1,3- | 20170 | orange |
| dihydroxybenzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′- | 27755 | black |
| naphthylazo]-1-hydroxy-7-aminonaphthalene-3,6- | | |
| disulfonic acid | | |
| 4′-[(4″-Sulfo-1″-phenylazo)-7′-sulfo-1′-naphthylazo]- | 28440 | black |
| 1-hydroxy-8-acetylaminonaphthalene-3,5-disulfonic | | |
| acid | | |
| Direct Orange 34, 39, 44, 46, 60 | 40215 | orange |
| Food Yellow | 40800 | orange |
| trans-β-Apo-8′-Carotenealdehyde (C₃₀) | 40820 | orange |
| trans-Apo-8′-Carotenic acid (C₃₀)-ethyl ester | 40825 | orange |
| Canthaxanthin | 40850 | orange |
| Acid Blue 1 | 42045 | blue |
| 2,4-Disulfo-5-hydroxy-4′-4″- | 42051 | blue |
| bis(diethylamino)triphenyl-carbinol | | |
| 4-[(4-N-Ethyl-p-sulfobenzylamino)phenyl-(4- | 42053 | green |
| hydroxy-2-sulfophenyl)(methylene)-1-(N-ethyl-N-p- | | |
| sulfobenzyl)-2,5-cyclohexadieneimine] | | |
| Acid Blue 7 | 42080 | blue |
| (N-Ethyl-p-sulfobenzylaminophenyl-(2-sulfophenyl)- | 42090 | blue |
| methylene-(N-ethyl-N-p- | | |
| sulfobenzyl)cyclohexadieneimine | | |
| Acid Green 9 | 42100 | green |
| Diethyldisulfobenzyldi-4-amino-2-chlorodi-2-methyl- | 42170 | green |
| fuchsonimmonium | | |
| Basic Violet 14 | 42510 | violet |
| Basic Violet 2 | 42520 | violet |
| 2′-Methyl-4′-(N-ethyl-N-m-sulfobenzyl)amino-4″-(N- | 42735 | blue |
| diethyl)-amino-2-methyl-N-ethyl-N-m- | | |
| sulfobenzylfuchsonimmonium | | |
| 4′-(N-Dimethyl)amino-4″-(N-phenyl)aminonaphtho- | 44045 | blue |
| N-dimethylfuchsonimmonium | | |
| 2-Hydroxy-3,6-disulfo-4,4′- | 44090 | green |
| bisdimethylaminonaphthofuchsinimmonium | | |
| Acid red | 45100 | red |
| 3-(2′-Methylphenylamino)-6-(2′-methyl-4′- | 45190 | violet |
| sulfophenylamino)-9-(2″-carboxyphenyl)xanthenium | | |
| salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminum complex | 58000 | red |
| 3-Oxypyrene-5,8,10-sulfonic acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis(o-sulfo-p-toluidine)anthraquinone | 61570 | green |
| Acid Blue 80 | 61585 | blue |
| Acid Blue 62 | 62045 | blue |
| N,N′-Dihydro-1,2,1′,2′-anthraquinoneazine | 69800 | blue |
| Vat Blue 6; Pigment Blue 64 | 69825 | blue |
| Vat Orange 7 | 71105 | orange |
| Indigo | 73000 | blue |
| Indigodisulfonic acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanines | 74260 | green |
| Natural Yellow 6,19; Natural Red 1 | 75100 | yellow |
| Bixin, Nor-Bixin | 75120 | orange |
| Lycopene | 75125 | yellow |
| trans-alpha, beta-or gamma-Carotene | 75130 | orange |
| Keto-and/or hydroxyl derivatives of carotene | 75135 | yellow |
| Guanine or pearlescent agents | 75170 | white |
| 1,7-Bis(4-hydroxy-3-methoxyphenyl)-1,6-heptadiene- | 75300 | yellow |
| 3,5-dione | | |
| Complex salt (Na,Al,Ca) of carminic acid | 75470 | red |
| Chlorophyll a and b; copper compounds of the | 75810 | green |
| chlorophylls and chlorophyllines | | |
| Aluminum | 77000 | white |
| Aluminum hydrate | 77002 | white |
| Water-containing aluminum silicates | 77004 | white |
| Ultramarine | 77007 | blue |
| Pigment Red 101 and 102 | 77015 | red |
| Barium sulfate | 77120 | white |
| Bismuth oxychloride and its mixtures with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268:1 | black |
| Chromic oxide | 77288 | green |
| Chromic oxide, water-containing | 77289 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxides and hydroxides | 77491 | red |
| Hydrated iron oxide | 77492 | yellow |
| Iron oxide | 77499 | black |
| Mixtures of iron(II) and iron(III) hexacyanoferrate | 77510 | blue |
| Pigment White 18 | 77713 | white |
| Manganese ammonium diphosphate | 77742 | violet |
| Manganese phosphate; Mn₃(PO₄)₂.7H₂O | 77745 | red |
| Silver | 77820 | white |
| Titanium dioxide and its mixtures with mica | 77891 | white |
| Zinc oxide | 77947 | white |
| 6,7-Dimethyl-9-(1′-D-ribityl)isoalloxazine, lactoflavin | | yellow |
| Caramel | | brown |
| Capsanthin, Capsorubin | | orange |
| Betanine | | red |
| Benzopyrilium salts, anthocyanines | | red |
| Aluminum, zinc, magnesium and calcium stearate | | white |
| Bromothymol Blue | | blue |
| Bromocresol Green | | green |
| Acid Red 195 | | red |

Oil-soluble natural dyes, such as, for example, paprika extracts, β-carotene and cochineal are furthermore advantageous.

Also advantageously used are pearlescent pigments, e.g. pearl essence (guanine/hypoxanthine mixed crystals from fish scales) and mother of pearl (ground mussel shells), monocrystalline pearlescent pigments such as, for example, bismuth oxychloride (BiOCl), layer substrate pigments, e.g. mica/metal oxide, silver-white pearlescent pigments from TiO₂, interference pigments (TiO₂, variable layer thickness), color luster pigments (Fe₂O₃) and combination pigments (TiO₂/Fe₂O₃, TiO₂/Cr₂O₃, TiO₂/Prussian blue, TiO₂/carmine).

Effect pigments within the context of the present invention are understood as meaning pigments which due to their refraction properties produce special optical effects. Effect pigments impart to the treated surface (skin, hair, mucous membrane) luster or glitter effects or can visually conceal unevenness of the skin and skin wrinkles by means of diffuse light scattering. As a particular embodiment of the effect pigments, interference pigments are preferred.

Particularly suitable effect pigments are, for example, mica particles which are coated with at least one metal oxide. Besides mica, a sheet silicate, silica gel and other SiO₂ modifications are also suitable as carriers. A metal oxide frequently used for coating is, for example, titanium oxide, to which, if desired, iron oxide can be admixed. By means of the size and shape (e.g. spherical, ellipsoidal, flat, even, uneven) of the pigment particles and by means of the thickness of the oxide coating, the reflection properties can be influenced. Other metal oxides, e.g. bismuth oxychloride (BiOCl), and the oxides of, for example, titanium, in particular the TiO₂ modifications anatase and rutile, aluminum, tantalum, niobium, zirconium and hafnium can also be used. Effect pigments can also be prepared using magnesium fluoride (MgF₂) and calcium fluoride (fluorspar, CaF₂).

The effects can be controlled both by means of the particle size and by means of the particle size distribution of the pigment ensemble. Suitable particle size distributions extend, for example, from 2-50 μm, 5-25 μm, 5-40 μm, 5-60 μm, 5-95 μm, 5-100 μm, 10-60 μm, 10-100 μm, 10-125 μm, 20-100 μm, 20-150 μm, and <15 μm. A wider particle size distribution, for example of 20-150 μm, produces glittering effects, whereas a narrower particle size distribution of <15 μm provides for a uniform silky appearance.

The preparations according to the invention comprise effect pigments preferably in amounts from 0.1-20% by weight, particularly preferably 0.5-10% by weight and especially preferably 1-5% by weight, in each case based on the total weight of the preparation.

The preferred inorganic photoprotective pigments are finely disperse or colloidally disperse metal oxides and metal salts, for example titanium oxide, zinc oxide, iron oxide, aluminum oxide, cerium oxide, zirconium oxide, silicates (talc) and barium sulfate. The particles should in this case have a mean diameter of less than 100 nm, preferably between 5 and 50 nm and particularly preferably between 15 and 30 nm, so-called ‘nanopigments’. They can have a spherical form, but it is also possible to use those particles which have an ellipsoidal shape or one which differs in another way from the spherical shape. The pigments can also be surface-treated, i.e. be present in hydrophilized or hydrophobicized form. Typical examples are coated titanium dioxides, such as, for example, titanium dioxide T 805 (Degussa) or Eusolex®T2000 (Merck). Possible hydrophobic coating agents are in this case especially silicones and in this context specifically trialkoxyoctylsilanes or dimethicones. Titanium dioxide and zinc oxide are particularly preferred.

The preferred inorganic particle substances are hydrophilic or amphiphilic. Advantageously, they can be superficially coated, in particular superficially treated to repel water. Examples of these are titanium oxide pigments coated with aluminum stearate, zinc oxide coated with dimethylpolysiloxane (dimethicone), boron nitride coated with dimethicone and titanium oxide coated with a mixture of dimethyl-polysiloxane and silica gel and hydrated aluminum oxide, titanium oxide coated with octylsilanol, or spherical polyalkylsesquioxane particles.

Organic photoprotective pigments are substances which are present in crystalline form at room temperature, which are able to absorb ultraviolet rays and to emit the absorbed energy again in the form of longer wavelength radiation, e.g. heat. A distinction is made between UVA filters and UVB filters. The UVA and UVB filters can be used both individually and in mixtures. The organic UV filters suitable according to the invention are selected from the derivatives, which are solid at room temperature, of dibenzoylmethane, cinnamic acid esters, diphenyl acid esters, benzophenone, camphor, p-aminobenzoic acid esters, o-aminobenzoic acid esters, salicylic acid esters, benzimidazoles, 1,3,5-triazines, monomeric and oligomeric 4,4-diarylbutadienecarboxylic acid esters and -carboxamides, ketotricyclo(5.2.1.0)decane, benzalmalonic acid esters, and any desired mixtures of the specified components. The organic UV filters may be oil-soluble or water-soluble. Particularly preferred oil-soluble UV filters according to the invention are (1-(4-tert-butylphenyl)-3-(4′-methoxyphenyl)propane-1,3-dione, 1-phenyl-3-(4′-isopropylphenyl)propane-1,3-dione, 3-(4′-methylbenzylidene)-D,L-camphor, 2-ethylhexyl 4-(dimethylamino)benzoate, 2-octyl 4-(dimethylamino)benzoate, amyl 4-(dimethylamino)benzoate, 2-ethylhexyl 4-methoxycinnamate, propyl 4-methoxycinnamate, isopentyl 4-methoxycinnamate, 2-ethylhexyl 2-cyano-3,3-phenylcinnamate (octocrylene), 2-ethylhexyl salicylate, 4-isopropylbenzyl salicylate, homomenthyl salicylate (3,3,5-trimethyloctylhexyl salicylate), 2-hydroxy-4-methoxybenzophenone, 2-hydroxy-4-methoxy-4′-methylbenzophenone, 2,2′-dihydroxy-4-methoxybenzophenone, di-2-ethylhexyl 4-methoxybenzmalonate, 2,4,6-trianilino(p-carbo-2′-ethyl-1′-hexyloxy)-1,3,5-triazine (octyltriazone) and dioctylbutamidotriazone, and any desired mixtures of the specified components.

Preferred water-soluble UV filters are 2-phenylbenzimidazole-5-sulfonic acid and its alkali metal, alkaline earth metal, ammonium, alkylammonium, alkanolammonium and glucammonium salts, sulfonic acid derivatives of benzophenones, preferably 2-hydroxy-4-methoxybenzophenone-5-sulfonic acid and its salts, sulfonic acid derivatives of 3-benzylidenecamphor, such as, for example, 4-(2-oxo-3-bornylidenemethyl)benzenesulfonic acid and 2-methyl-5-(2-oxo-3-bornylidene)sulfonic acid and their salts.

In the preparations according to the invention, the inorganic and organic photoprotective pigments are present in amounts of preferably 0.1 to 30% by weight, particularly preferably 1 to 20% by weight and especially preferably 2 to 15% by weight, in each case based on the total weight of the preparation.

The preparations according to the invention can contain particulate inorganic or organic adsorbents having mean particle diameters of 1-100 μm. The adsorbents are selected from pyrogenic silicas, e.g. the aerosol types, precipitated silicas, silica gels, silicon dioxide, clays, e.g. bentonite or kaolin, magnesium aluminum silicates, e.g. talc and boron nitride, optionally modified starches and starch derivatives, cellulose powders, lactoglobulin derivatives, polymer powders of polyolefins, polycarbonates, polyurethanes, polyamides, polyesters, polystyrenes, polyacrylates, (meth)acrylate or (meth)acrylate-vinylidene copolymers, which can be crosslinked, Teflon or silicones, and mixtures of the substances mentioned.

The preparations according to the invention can contain abrasive components, for example ground plant parts such as almond bran or wheat bran, crystalline cellulose, hydrogenated jojoba oil, polymer beads, preferably of polyethylene or polyamide-11, having mean diameters of 90-600 μm, and of active ingredient-containing micro- or millicapsules, which contain petrochemical polymers (e.g. from polyamide such as nylon-11) and/or biopolymers such as gelatin, pectin, plant gums, alginates and carrageenan. Almond bran, wheat bran, hydrogenated jojoba oil and polyethylene beads are preferably used.

In a preferred embodiment of the invention, the preparations in question are make-up, eye make-up, mascara, eyeliner and blusher, distinguished by particular water resistance, color brilliance, pearlescent effect, good skin sensory properties and good spreadability of the compositions on the skin.

In a further preferred embodiment of the invention, the preparations in question are nail varnish having excellent gloss effects.

In a further preferred embodiment, the preparations according to the invention comprise one or more UV photoprotective filters. These preparations according to the invention are preferably sunscreen compositions. The sunscreen compositions are preferably in the form of sprays, sticks, pastes, gels or lotions.

Suitable UV filters are preferably 4-aminobenzoic acid; 3-(4′-trimethylammonium)benzylideneboran-2-one methylsulfate; 3,3,5-trimethylcyclohexyl salicylate; 2-hydroxy-4-methoxybenzophenone; 2-phenylbenzimidazole-5-sulfonic acid and its potassium, sodium and triethanolamine salts; 3,3′-(1,4-phenylenedimethine)bis(7,7-dimethyl-2-oxobicyclo-[2.2.1]-heptane-1-methanesulfonic acid and its salts; 1-(4-tert-butylphenyl)-3-(4-methoxyphenyl)propane-1,3-dione, 3-(4′-sulfo)benzylideneboman-2-one and its salts; 2-ethylhexyl 2-cyano-3,3-diphenylacrylate; polymer of N-[2(and 4)-(2-oxoborn-3-ylidene-methyl)benzyl]acrylamide; 2-ethylhexyl 4-methoxycinnamate; ethoxylated ethyl 4-aminobenzoate; isoamyl 4-methoxycinnamate; 2,4,6-tris[p-(2-ethylhexyloxy-carbonyl)anilino]-1,3,5-triazine; 2-(2H-benzotriazol -2-yl)-4-methyl-6-(2-methyl-3-(1,3,3,3-tetramethyl-1-(trimethylsilyloxy)disiloxanyl)propyl)phenol; 4,4′-[(6-[4-((1,1-dimethylethyl)aminocarbonyl)phenylamino]-1,3,5-triazin-2,4-yl)d iimino]bis(2-ethylhexyl benzoate); 3-(4′-methylbenzylidene)-D,L-camphor; 3-benzylidenecamphor; 2-ethylhexyl salicylate; 2-ethylhexyl 4-dimethylaminobenzoate; hydroxy-4-methoxybenzophenone-5-sulfonic acid (sulisobenzonum) and the sodium salt; and/or 4-isopropylbenzyl salicylate, N,N, N-trimethyl-4-(2-oxoborn-3-ylidenemethyl)anilium methylsulfate, homosalate (INN), oxybenzone (INN), 2-phenylbenzimidazole-5-sulfonic acid and its Na, K, and triethanolamine salts, alpha-(2-oxoborn-3-ylidene)toluene-4-sulfonic acid and its salts, octyl methoxycinnamate, isopentyl 4-methoxycinnamate, isoamyl p-methoxycinnamate, 2,4,6-trianilino-(p-carbo-2′-ethylhexyl-1′-oxy)-1,3,5-triazine (octyltriazone), phenol, 2-2(2H-benzotriazol-2-yl)-4-methyl-6-(2-methyl-3-(1,3,3,3-tetramethyl-1-(trimethylsilyl)oxy)disiloxanyl)propyl (drometriazole trisiloxane)benzoic acid, 4,4-((6-(((1,1-dimethylethyl)amino)-carbonyl)phenyl)amino)-1,3,5-triazine-2,4-diyl)diimino)bis, bis(2-ethylhexyl))ester)benzoic acid, 3-(4′-methylbenzylidene)-d-1-camphor (4-methylbenzylidenecamphor), 3-benzylidenecamphor, 2-ethylhexyl salicylate (octyl salicylate), ethyl-2-hexyl 4-dimethylaminobenzoate (octyl dimethyl PABA), 2-hydroxy-4-methoxybenzophenone-5-sulfonic acid (benzophenone-5) and the Na salt, 2,2′-methylenebis-6-(2H-benzotriazol-2-yl)-4-(tetramethylbutyl)-1,1,3,3-phenol, sodium salt of 2-2′-bis(1,4-phenylene)-1H-benzimidazole-4,6-disulfonic acid, (1,3,5)-triazine-2,4-bis((4-(2-ethylhexyloxy)-2-hydroxy)phenyl)-6-(4-methoxyphenyl), 2-ethylhexyl 2-cyano-3,3-diphenyl-2-propenoate, glyceryl octanoate, di-p-methoxycinnamic acid, p-aminobenzoic acid and ester, 4-tert-butyl-4′-methoxyd ibenzoylmethane, 4-(2-β-glucopyranoxy)propoxy-2-hydroxybenzophenone, octyl salicylate, methyl 2,5-diisopropylcinnamate, cinoxate, dihydroxydimethoxybenzophenone, disodium salt of 2,2′-dihydroxy-4,4′-dimethoxy-5,5′-disulfobenzo-phenone, dihydroxybenzophenone, 1-(3,4-dimethoxyphenyl)-4,4-dimethyl-1,3-pentanedione, 2-ethylhexyl dimethoxybenzylidene dioxoimidazolidine propionate, tetrahydroxybenzophenone, terephthalylidenedicamphorsulfonic acid, 2,4,6-tris[4-2-ethylhexyloxycarbonyl)anilino]-1,3,5-triazine, methyl bis(trimethylsiloxy)silyl isopentyltrimethoxycinnamate, amyl p-dimethylaminobenzoate, 2-ethylhexyl p-dimethylaminobenzoate, isopropyl p-methoxycinnamate/diisopropyl cinnamate, 2-ethylhexyl p-methoxycinnamate, 2-hydroxy-4-methoxybenzophenone, 2-hydroxy-4-methoxybenzophenone-5-sulfonate and the trihydrate, 2-hydroxy-4-methoxybenzophenone-5-sulfonate, Na salt and phenylbenzimidazolesulfonic acid.

Further suitable UV filters are chosen from the derivatives, selected which are solid at room temperature, of dibenzoylmethane, cinnamic acid esters, diphenyl acid esters, benzophenone, camphor, p-aminobenzoic acid esters, o-aminobenzoic acid esters, salicylic acid esters, benzimidazoles, 1,3,5-triazines, monomeric and oligomeric 4,4-diarylbutadienecarboxylic acid esters and -carboxamides, ketotricyclo(5.2.1.0)decane, benzalmalonic acid esters, and any desired mixtures of the specified components. The organic UV filters may be oil-soluble or water-soluble. Oil-soluble UV filters which are particularly preferred according to the invention are 1-phenyl-3-(4′-isopropylphenyl)-propane-1,3-dione, 2-octyl(dimethylamino)benzoate, amyl 4-(dimethylamino)benzoate, 2-ethylhexyl 4-methoxycinnamate, propyl 4-methoxycinnamate, isopentyl 4-methoxycinnamate, 2-ethylhexyl 2-cyano-3,3-phenylcinnamate (octocrylene), 4-isopropylbenzyl salicylate, homomenthyl salicylate (3,3,5-trimethyloctylhexyl salicylate), 2-hydroxy -4-methoxybenzophenone, 2-hydroxy-4-methoxy-4′-methyl-benzophenone, 2,2′-dihydroxy-4-methoxybenzophenone, di-2-ethylhexyl 4-methoxybenzmalonate, 2,4,6-trianilino(p-carbo-2′-ethyl-1′-hexyloxy)-1,3,5-triazine (octyltriazone) and dioctylbutamidotriazone, and any desired mixtures of the specified components. Preferred water-soluble UV filters are 2-phenylbenzimidazole-5-sulfonic acid and its alkali metal, alkaline earth metal, ammonium, alkylammonium, alkanblammonium and glucammonium salts, sulfonic acid derivatives of benzophenones, preferably 2-hydroxy-4-methoxybenzophenone-5-sulfonic acid and its salts, sulfonic acid derivatives of 3-benzylidenecamphor, such as, for example, 4-(2-oxo-3-bornylidenemethyl)benzenesulfonic acid and 2-methyl-5-(2-oxo-3-bornylidene)sulfonic acid and their salts.

The preparations according to the invention, for example the sunscreen compositions, comprise UV photoprotective filters in the amounts by weight of preferably 0.1 to 10%, particularly preferably from 0.5 to 8% and especially preferably from 1 to 5%, based on the finished preparations.

The sunscreen compositions can comprise, as UV photoprotective filter, however, for example, also the inorganic UV absorbers and photoprotective pigments specified under the group of pigments listed above. These are present in the preparations according to the invention preferably in amounts of from 0.1 to 30% by weight, particularly preferably in amounts of from 0.5 to 15% by weight and especially preferably in amounts of from 1.0 to 10% by weight, in each case based on the total weight of the preparations.

In a further preferred embodiment, the preparations according to the invention, for example the sunscreen compositions, comprise one or more antioxidants.

Advantageously, the antioxidants are chosen from the group consisting of amino acids (e.g. glycine, histidine, tyrosine, tryptophan) and their derivatives, imidazoles (e.g. urocaninic acid) and their derivatives, peptides such as D,L-carnosine, D-carnosine, L-carnosine and their derivatives (e.g. anserine), carotenoids, carotenes (e.g. α-carotene, β-carotene, lycopene) and their derivatives, chlorogenic acid and its derivatives, lipoic acid and its derivatives (e.g. dihydrolipoic acid), aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and their salts, dilauryl thiodipropionate, distearyl thiodipropionate, thiodipropionic acid and its derivatives (esters, ethers, peptides, lipids, nucleotides, nucleosides and salts) and sulfoximine compounds (e.g. buthionine sulfoximine, homocysteine sulfoximine, buthionine sulfone, penta-, hexa-, heptathionine sulfoximine) in very low tolerable doses, furthermore (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. γ-linolenic acid, linoleic acid, oleic acid), folic acid and its derivatives, ubiquinone and ubiquinol and their derivatives, vitamin C and derivatives (e.g. ascorbylpalmitate, Mg ascorbylphosphate, ascorbylacetate), tocopherols and derivatives (e.g. vitamin E acetate), vitamin A and derivatives (vitamin A palmitate) and coniferyl benzoate of benzoin resin, rutic acid and its derivatives, α-glycosylrutin, ferulic acid, furfurylideneglucitol, carnosine, butylhydroxytoluene, butylhydroxyanisole, nordihydroguaiaretic acid, trihydroxybutyrophenone, uric acid and its derivatives, mannose and its derivatives, zinc and its derivatives (e.g. ZnO, ZnSO₄), selenium and its derivatives (e.g. selenomethionine), stilbenes and their derivatives (e.g. stilbene oxide, trans-stilbene oxide), superoxide dismutase and the derivatives suitable according to the invention (salts, esters, ethers, sugars, nucleotides, nucleosides, peptides and lipids) of the substances mentioned.

Water-soluble antioxidants can be used particularly advantageously within the meaning of the present invention.

The antioxidants are able to protect the skin and the hair from oxidative stress. Preferred antioxidants here are vitamin E and its derivatives and vitamin A and its derivatives.

The amount of the one or more antioxidants in the preparations according to the invention is preferably 0.001 to 30% by weight, particularly preferably 0.05 to 20% by weight and especially preferably 1 to 10% by weight, based on the total weight of the preparations.

If vitamin E and/or its derivatives is/are the antioxidant(s), it is advantageous to choose its/their respective concentrations from the range from 0.001 to 10% by weight, based on the total weight of the preparation. If vitamin A, or vitamin A derivatives, or carotenes or their derivatives is/are the antioxidant(s), it is advantageous to choose its/their respective concentrations from the range from 0.001 to 10% by weight, based on the total weight of the preparation.

In a particularly preferred embodiment of the invention, the cosmetic or pharmaceutical preparations comprise antioxidants selected from superoxide dismutase, tocopherol (vitamin E) and ascorbic acid (vitamin C).

In a further preferred embodiment, the preparations according to the invention are deodorants and antiperspirants which comprise one or more substances chosen from antimicrobially effective substnaces, astringents and deodorizing substances. These preparations are preferably present in the form of sprays, sticks, pastes, gels or lotions.

Preferably, suitable antimicrobial active compounds are cetyltrimethylammonium chloride, cetylpyridinium chloride, benzethonium chloride, diisobutylethoxyethyl-dimethylbenzylammonium chloride, sodium N-laurylsarcosinate, sodium N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether (triclosan), phenoxyethanol, 1,5-pentanediol, 1,6-hexanediol, 3,4,4′-trichlorocarbanilide (triclocarban), diaminoalkylamide, for example L-lysine hexadecylamide, citrate heavy-metal salts, salicylates, piroctose, in particular zinc salts, pyrithiones and their heavy-metal salts, in particular zinc pyrithione, zinc phenolsulfate, farnesol and combinations of these active substances.

The preparations according to the invention comprise the antimicrobial active compounds preferably in amounts of up to 50% by weight, particularly preferably in amounts of from 0.01 to 10% by weight and especially preferably in amounts of from 0.1 to 10% by weight, based on the finished preparations.

Preferred astringents are oxides, preferably magnesium oxide, aluminum oxide, titanium dioxide, zirconium dioxide and zinc oxide, hydrated oxides, preferably hydrated aluminum oxide (boehmite) and hydroxides, preferably of calcium, magnesium, aluminum, titanium, zirconium or zinc.

The preparations according to the invention comprise the astringent active compounds preferably in amounts of from 0 to 50% by weight, particularly preferably in amounts of from 0.01 to 10% by weight and especially preferably in amounts of from 0.1 to 10% by weight, based on the finished preparations.

Allantoin and bisabolol are preferred as deodorizing substances. These are preferably used in amounts of from 0.0001 to 10% by weight.

In a further preferred embodiment, the preparations according to the invention are peels. These are preferably present in the form of peel creams or gels for cleansing and smoothing the skin.

In a further preferred embodiment, the preparations according to the invention are dental care compositions, in particular dental care compositions which comprise the abovementioned homopolymer and copolymer waxes in micronized form as abrasive component. The dental care compositions can be in the form of toothpaste, tooth cream or tooth gel.

In a further preferred embodiment, the preparations according to the invention are hair removal compositions.

As further auxiliaries and additives, the preparations according to the invention can comprise other pulverulent substances, filling materials, cationic polymers, film-formers, thickeners and dispersants, superfatting agents, moisturizing agents, stabilizers, biogenic active ingredients, glycerol, preservatives, pearlizing agents, fragrances, solvents, opacifiers, further waxes, further protein derivatives such as gelatin, collagen hydrolyzates, natural and synthetic polypeptides, egg yolk, lecithin, lanolin and lanolin derivatives, fatty alcohols, silicones, cooling agents, for example methyl acetate, substances having keratolytic and keratoplastic action, enzymes and carrier substances.

In addition, the filling materials used can be SiO₂, silica, ZnO, kaolin, SiO₂-modified kaolin, polytetrafluoroethylene, nylon, talc, mica, polymethyl methacrylate, polyethylene, polyethers, polycarbonates, polyvinyl chloride, polystyrene, polyamides, polyurethanes, polyacrylates, natural polymers, silk powder, microcrystalline cellulose, natural organic compounds such as encapsulated or unencapsulated grain meal and mixtures thereof.

The cationic polymers available are those known under the INCI name “Polyquaternium”, in particular polyquaternium-31, polyquaternium-16, polyquaternium-24, polyquaternium-7, polyquaternium-22, polyquaternium-39, polyquaternium-28, polyquaternium-2, polyquaternium-10, polyquaternium-11, as well as polyquaternium 37 & mineral oil & PPG trideceth (Salcare SC95), PVP-dimethylaminoethyl methacrylate copolymer, guar hydroxypropyltriammonium chlorides, and also calcium alginate and ammonium alginate. Likewise suitable are cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethylenimines; cationic silicone polymers, such as, for example, amidomethicones; copolymers of adipic acid and dimethylaminohydroxypropyldiethylenetriamine; polyaminopolyamide and cationic chitin derivatives, such as, for example, chitosan.

Suitable silicone compounds are, for example, dimethylpolysiloxane, methylphenylpolysiloxanes, cyclic silicones and amino-, fatty acid-, alcohol-, polyether-, epoxy-, fluoro- and/or alkyl-modified silicone compounds, and also polyalkylsiloxanes, polyalkylarylsiloxanes, polyethersiloxane copolymers, as described in U.S. Pat. No. 5,104,645 and the specifications cited therein, which can be present either in liquid or resinous form at room temperature.

Suitable film-formers are, depending on the intended use, salts of phenylbenzimidazolesulfonic acid, water-soluble polyurethanes, for example C₁₀-polycarbamyl polyglycerol esters, but also polyvinyl alcohol, polyvinylpyrrolidone, and also copolymers, for example vinylpyrrolidone/vinyl acetate copolymer, water-soluble acrylic acid polymers/copolymers or their esters or salts, for example partial ester copolymers of acrylic/methacrylic acid and polyethylene glycol ethers of fatty alcohols, such as acrylate/steareth-20 methacrylate copolymer, water-soluble cellulose, for example hydroxymethylcellulose, hydroxyethylcellulose, hydroxypropylcellulose, water-soluble quaterniums, polyquaterniums, carboxyvinyl polymers, such as carbomers and their salts, polysaccharides, for example polydextrose and glucan.

Superfatting agents which can be used are substances such as, for example, lanolin, lecithin, polyethoxylated lanolin derivatives, lecithin derivatives, polyol fatty acid esters, monoglycerides and fatty acid alkanolamides, where the latter can simultaneously serve as foam stabilizers.

Moisturizing substances available are, for example, isopropyl palmitate, glycerol and/or sorbitol, which are preferably used in the amounts of from 0.1 to 50% by weight.

Biogenic active ingredients which can be used are, for example, plant extracts, for example, Aloe vera, and vitamin complexes, Bisabolol®, Allantoin®, Phytantriol®, Panthenol®, AHA acids, local anesthetics, antibiotics, antiinflammatories, antiallergics, corticosteroids, sebostatics, phanthenol, allantoin and proteins.

Suitable preservatives are, for example, phenoxyethanol, diazolidinylurea, parabens, pentanediol, butylated hydroxytoluene, butylated hydroxyanisole or sorbic acid. They are preferably used in the amounts of from 0.001 to 5% by weight, particularly preferably from 0.01 to 3% by weight and especially preferably from 0.1 to 2% by weight, based on the finished preparations.

Dyes which can be used are the substances suitable and permitted for cosmetic and pharmaceutical purposes.

Substances preferably suitable as pearlescence-imparting components are fatty acid monoalkanolamides, fatty acid dialkanolamides, monoesters or diesters of alkylene glycols, in particular ethylene glycol and/or propylene glycol or its oligomers, with higher fatty acids, such as e.g. palmitic acid, stearic acid and behenic acid, monoesters or polyesters of glycerol with carboxylic acids, fatty acids and their metal salts, ketosulfones or mixtures of the compounds mentioned. Ethylene glycol distearates and/or polyethylene glycol distearates having on average 3 glycol units are particularly preferred.

If the preparations according to the invention contain pearlescence-imparting compounds, these are preferably present in the preparations according to the invention in an amount of from 0.1 to 15% by weight and particularly preferably in an amount of from 1 to 10% by weight.

Suitable fungicidal active compounds are preferably ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, Zn pyrithione and octopyrox.

Particularly suitable thickeners and dispersants are ethylene glycol esters of fatty acids having 14 to 22, particularly preferably 16 to 22, carbon atoms, in particular mono- and diethylene glycol stearate. Likewise preferably suitable are stearic monoethanolamide, stearic diethanolamide, stearic isopropanolamide, stearic monoethanolamide stearate, stearyl stearate, cetyl palmitate, glyceryl stearate, stearamide diethanolamide distearate, stearamide monoethanolamide stearate, N,N-dihydrocarbyl-(C₁₂-C₂₂)-amidobenzoic acid and its soluble salts, N,N-dihydro-carbyl-(C₁₆-C₁₈)-amidobenzoic acid and its soluble salts and N,N-di(C₁₆-C₁₈)-amidobenzoic acid and its derivatives. Furthermore particularly suitable are polyacrylates and carbomers, in particular those water-soluble or water-swellable copolymers based on acrylamidoalkylsulfonic acids and N-vinylcarboxylic acid amides.

In order to increase the color intensity, the preparations according to the invention can comprise the carriers customary in cosmetic systems, in particular benzyl alcohol, vanillin (4-hydroxy-3-methoxybenzaldehyde), isovanillin, p-hydroxyanisole, 3-hydroxy-4-methoxybenzaldehyde, 2-phenoxyethanol, salicylaldehyde, 3,5-di-hydroxybenzaldehyde, 3,4-dihydroxybenzaldehyde, 4-hydroxyphenylacetamide, methyl p-hydroxybenzoate, p-hydroxybenzaldehyde, m-cresol, hydroquinone monomethyl ether, o-fluorophenol, m-fluorophenol, p-fluorophenol, 2-(2′-hydroxy-phenoxy)ethanol, 3,4-methylenedioxyphenol, resorcinol monomethyl ether, 3,4-dimethoxyphenol, 3-trifluoromethylphenol, resorcinol monoacetate, ethylvanillin, 2-thiopheneethanol, butyl lactate and butyl glycolate. Preparations according to the invention comprising phenoxyethanol and/or benzyl alcohol are particularly advantageous with a synergistic action.

Suitable solubilizers are in principle all mono- or polyhydric alcohols and ethoxylated alcohols. Preferably, alcohols having 1 to 4 carbon atoms, such as, for example, ethanol, propanol, isopropanol, n-butanol and isobutanol, glycerol and their mixtures are used. Furthermore preferred are polyethylene glycols having a relative molecular mass of below 2000. Particularly preferred are polyethylene glycols having a relative molecular mass of between 200 and 600 in amounts of up to 45% by weight and polyethylene glycols having a relative molecular mass of between 400 and 600 in amounts of from 0.5 to 15% by weight. Further suitable solvents are, for example, triacetin (glycerol triacetate) and 1-methoxy-2-propanol.

The stabilizers used can be metal salts of fatty acids, such as, for example, magnesium stearate, aluminum stearate and/or zinc stearate.

The preparations according to the invention can be mixed with conventional ceramides, pseudoceramides, fatty acid N-alkylpolyhydroxyalkylamides, cholesterol, cholesterol fatty acid esters, fatty acids, triglycerides, cerebrosides, phospholipids and similar substances as care additives.

The scent or perfume oils used can be individual odorant compounds, e.g. the synthetic products of the type consisting of the esters, ethers, aldehydes, ketones, alcohols and hydrocarbons. Odorant compounds of the type consisting of the esters are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenyl glycinate, allylcyclohexyl propionate, styrallyl propionate and benzyl salicylate. The ethers include, for example, benzyl ethyl ether, the aldehydes, for example, the linear alkanals having 8 to 18 carbon atoms, citral, citronellal, citronellyloxyacetaldehyde, hydroxycitronellal, lilial and bourgeonal, the ketones, for example, the ionones, alpha-isomethylionone and methyl cedryl ketone, the alcohols anethol, citronellol, eugenol, geraniol, linalool, phenylethyl alcohol and terpineol, the hydrocarbons include mainly the terpenes and balsams. Preferably, mixtures of various odorants are used, which together produce a pleasant scent note.

Perfume oils can also contain natural odorant mixtures, such as are accessible from vegetable or animal sources, e.g. pine, citrus, jasmine, lily, rose or ylang-ylang oil. Ethereal oils of relatively low volatility, which are usually used as flavoring components, are also suitable as perfume oils, e.g. sage oil, camomile oil, oil of cloves, melissa oil, mint oil, oil of cinnamon leaves, linden blossom oil, oil of juniper berries, vetiver oil, olibanum oil, galbanum oil and ladanum oil.

Further additives can be silicone compounds, preferably dimethylpolysiloxane, methylphenylpolysiloxanes, cyclic silicones and also amino-, fatty acid-, alcohol-, polyether-, epoxy-, fluoro- and/or alkyl-modified silicone compounds, for example alkylsilicones: SilCare® Silicone 41M10, SilCare® Silicone 41M15, SilCare® Silicone 41M20, SilCare® Silicone 41M30 (Clariant), alkyltrimethicones: SilCare® 31M30, SilCare® 31M40, SilCare® 31M 50, SilCare® 31M 60 (Clariant), phenyltrimethicones: SilCare® 15M30, SilCare® 15M40, SilCare® 15M50, SilCare® 15M60 (Clariant), polyalkylarylsiloxanes and polyethersiloxane copolymers.

The preparations according to the invention can comprise the abovementioned silicone compounds preferably in the amounts by weight of from 0.1 to 20% by weight, particularly preferably from 0.2 to 15% by weight and especially preferably from 0.5 to 10% by weight, based on the finished preparations.

The preparations customarily have a pH in the range from 2 to 12 and preferably in a range from 3 to 8.

The following examples and applications are intended to illustrate the invention in greater detail, but without restricting it thereto (all percentages are percentages by weight).

The metallocene-polyolefin waxes listed in Table 1 were produced according to the method given in EP-A-0 571 882 from the monomers ethylene and/or propylene. The weight fraction of the monomers is given in Table 1.

TABLE 1
Composition of the polyolefin waxes

| | Ethylene [% by | Propylene |
|---|---|---|
| Polyolefin wax | wt.] | [% by wt.] |
| 1 | 100 | 0 |
| 2 | 95 | 5 |
| 3 | 0 | 100 |
| 4 | 10 | 90 |

The dropping point, the viscosity, the weight-average molecular weight Mw and the density of the polyolefin waxes 1 to 4 was determined. The results are given in Table 2.

TABLE 2
Softening/dropping point, viscosity, weight-average
molecular weights and density of the polyolefin waxes

| | | | Viscosity at | Weight-average | |
|---|---|---|---|---|---|
| Polyolefin | | Dropping | 170° C. | molecular weight | Density |
| wax | Product type | point [° C.] | [mPa s] | Mw [g/mol] | [g/cm³] |
| 1 | Ethylene | 128 | 100 | 4200 | 0.98 |
| | homopolymer wax | | | | |
| | (metallocene) | | | | |
| 2 | Ethylene- | 109 | 500 | 6800 | 0.93 |
| | propylene | | | | |
| | copolymer wax | | | | |
| | (metallocenes) | | | | |
| 3 | Propylene | 150 | 120 | 4500 | 0.89 |
| | homopolymer wax | | | | |
| | (metallocene) | | | | |
| 4 | Propylene- | 90 | 150 | 5800 | 0.88 |
| | ethylene | | | | |
| | copolymer wax | | | | |
| | (metallocene) | | | | |

The paste hardness of a preparation comprising polyolefin wax 2 and the paste hardness of 2 corresponding preparations which comprise standard commercial waxes instead of polyolefin wax 2 were determined and compared with one another. The results are given in Table 3.

TABLE 3
Paste hardness of a polyolefin wax produced by means of
metallocene catalysis compared with polyolefin waxes
prepared using a Ziegler-Natta catalyst (Licowax PE 520)
or produced by free-radical polymerization under high-
pressure conditions (A-C 8)

| | | | |
|---|---|---|---|
| Paraffin wax | 17.1 | 17.1 | 17.1 |
| Ozokerite 2089 | 0.9 | 0.9 | 0.9 |
| Polyolefin wax 2 | 7 | — | — |
| Licowax PE 520 | — | 7 | — |
| A-C 8 | — | — | 7 |
| White spirit | ad 100 | ad 100 | ad 100 |
| Paste hardness [g/cm²] 25° C. | 2460 | 420 | 1880 |
| Paste hardness [g/cm²] 40° C. | 530 | <50 | 475 |
| Paste hardness [g/cm²] 50° C. | 310 | <50 | 120 |
| Pour temperature [° C.] | 66 | 70 | 70 |

The results of Table 3 show that polyolefin waxes produced using metallocene catalysis are very well suited for producing sticks and pencils, for example lipsticks.

### FORMULATION EXAMPLES

### Example 1

### W/O Cream

| | | |
|---|---|---|
| A | Hostacerin ® DGI | 6.0% |
| | Magnesium stearate | 1.0% |
| | Mineral oil, low viscosity | 5.0% |
| | Vaseline | 10.0% |
| | Cetiol ® V | 5.0% |
| B | 1,2-Propylene glycol | 3.0% |
| | Water dist. | ad 100% |
| | Preservative | q.s. |
| C | Fragrance | 0.4% |
| D | Polyolefin wax 1, micronized | 3.0% |
| | Preparation: | |
| I | Melting of A at 80° C. | |
| II | Heating of B to 80° C. | |
| III | Stirring II into I | |
| IV | Stirring until a temperature of 35° C. has been reached | |
| V | Addition of C to IV at 35° C. | |
| VI | Stirring of D into V at room temperature | |

### Example 2

### O/W Cream

| | | | |
|---|---|---|---|
| A | Hostacerin ® DGI | | 2.0% |
| | SilCare ® Silicone 31M30 | Clariant | 4.0% |
| | Perliquidum | | 4.0% |
| | Polyolefin wax 2 | | 2.0% |
| | Eutanol G | Clariant | 4.0% |
| | Isopropyl palmitate | Clariant | 4.0% |
| | Carbopol 980 | | 0.7% |
| B | Hostapon ® KCG | | 0.6% |
| | Sodium hydroxide solution (10% in water) | | 2.1% |
| | Preservative | | q.s. |
| | Fragrance | | 0.4% |
| | Water dist. | | ad 100% |
| | Preparation: | | |
| I | Heating of A to 80° C. | | |
| II | Heating of B to 80° C. | | |
| III | Emulsifying by slowly stirring B into A. | | |

### Example 3

### Antiperspirant

| | | | |
|---|---|---|---|
| A | Locron ® L | Clariant | 10.0% |
| | Ethanol | | 50.0% |
| | Farnesol | | 0.5% |
| | Fragrance | | 0.2% |
| | Polyolefin wax 1 | | 0.5% |
| | Water dist. | | ad 100% |
| | Extrapon Avocado special | | 1.5% |
| | Preparation: | | |
| | Mixing of components A | | |

### Example 4

### W/O Antiperspirant Cream

| | | |
|---|---|---|
| A | Abil EM90 | 2.0% |
| | Abil B8839 | 20.0% |
| | Polyolefin wax 1 | 2.0% |
| B | Aloxicoll L | 17.0 |
| | Water dist. | ad 100% |
| | Perfume oil | q.s. |
| | Preservative | q.s. |
| | Preparation: | |
| I | Slowly add phase B to phase A with stirring at room | |
| | temperature. | |
| II | Homogenize | |

### Example 5

### Deodorant Stick

| | | | |
|---|---|---|---|
| A | OCTOPIROX ® | (Clariant) | 0.1% |
| | Polyolefin wax 2 | | 3.0% |
| | Propylene glycol | | 71.0% |
| | Rewoderm 66E | | 5.0% |
| | Sodium stearate | | 5.0% |
| | GENAPOL ® HS 020 | (Clariant) | 1.0% |
| | Water dist. | | ad 100% |
| | Preparation: | | |

| | |
|---|---|
| I | Mixing of components A at 50° C. |
| | and stirring until the solution is clear |
| II | Bottling and cooling |

### Example 6

### Alcohol-Free Deodorant Roll-On (Opaque)

| | | |
|---|---|---|
| A | Tegodeo CW 90 | 2.0% |
| | Polyethylene glycol(3) lauryl ether | 1.0% |
| | Triethanolamine | 1.0% |
| B | Polyolefin wax 2 | 1.2% |
| | Water dist. | ad 100% |
| C | Tagat R 40 | 3.0% |
| | Perfume oil | q.s. |
| | Preservative | q.s. |
| D | Citric acid (50% strength in water) | 0.2% |
| | Preparation: | |
| I | Heat phases A and B separately to 80° C. | |
| II | Stir phase B into phase A and homogenize | |
| III | Cool with slow stirring | |
| IV | At 30° C., add phase C | |
| V | Adjust the pH using phase D | |

### Example 7

### Foundation

| | | | |
|---|---|---|---|
| A | Nexbase ® 2004 FG | | 9.0% |
| | Myritol ® 318 | | 5.0% |
| | Almond Oil | | 4.0% |
| | SilCare ® Silicone 31M40 | (Clariant) | 4.0% |
| | SilCare ® Silicone 41M15 | (Clariant) | 3.0% |
| | Genapol ® HS020 | (Clariant) | 1.6% |
| | Genapol ® HS200 | (Clariant) | 2.4% |
| | Polyolefin wax 1 | | 2.0% |
| B | Vanclay ® | | 1.5% |
| | Talc | | 3.0% |
| | Iron Oxide Pigments | | 7.9% |
| C | Glycerol | | 5.0% |
| | Water dist. | | ad 100% |
| | Aristoflex ® AVC | (Clariant) | 0.4% |
| D | Fragrance | | q.s. |
| | Nipaguard ® PDU | (Clariant) | q.s. |
| | Preparation: | | |

| | |
|---|---|
| I | Mixing and melting of components A at 70° C. |
| II | Addition of B to I at 70° C. with stirring |
| III | Mixing of C until Aristoflex ® AVC has dissolved |
| | and heating to 70° C. |
| IV | Addition of C to II with stirring and homogenization. |
| V | Addition of D to IV at <40° C. |

### Example 8

### Mascara

| | | | |
|---|---|---|---|
| A | Tylose ® H 4000 G4 | | 0.7% |
| | 1,2-Propylene glycol | | 1.0% |
| | Water dist. | | ad 100% |
| B | Triethanolamine 99% | | 1.2% |
| C | Stearic acid | | 3.0% |
| | SilCare ® Silicone 41M15 | (Clariant) | 1.0% |
| | SilCare ® Silicone 31M40 | (Clariant) | 2.0% |
| | Tegocare ® 450 | | 4.0% |
| | Nexbase ® 2006 | | 2.0% |
| | Beeswaxes | | 2.5% |
| | Candelilla wax | | 2.5% |
| | Polyolefin wax 1 | | 3.5% |
| D | Pigments | | 10.0% |
| E | Nipagin ® M | (Clariant) | 0.2% |
| | Nipasol ® M | (Clariant) | 0.1% |
| F | Fragrance | | q.s |
| | Preparation: | | |
| I | Swell components A at room temperature | | |
| | with stirring; heat to 85° C. | | |
| II | Addition of B to A and stir | | |
| III | Melting of components C to 85° C. | | |
| IV | Addition of D to III with stirring at 85° C. | | |
| V | Addition of II to IV with vigorous stirring | | |
| | (15 minutes at 85° C., further | | |
| | 15 minutes without heating) | | |
| VI | Addition of E and F to V at 35 to 40° C. | | |
| VII | Bottling at 35° C. to 40° C. | | |

### Example 9

### Dental Cream

| | | |
|---|---|---|
| A | Water dist. | ad 100% |
| | Glycerol | 20.0% |
| | Sodium benzoate | 2.5% |
| | Saccharin (10% strength) | 1.0% |
| | Aroma oil | 1.0% |
| | Cosmenyl Green GG | 0.005% |
| | Medialan ® LD | 6.6% |
| B | Tylose H 20 | 2.4% |
| C | Aerosil ® 300 | 2.0% |
| | Polyolefin wax 1 micronized | 32.0% |
| | Preparation: | |
| I | Mixing and stirring of components A | |
| II | Stirring of B into I | |
| III | Successive stirring of components C into II | |

### Example 10

### Dental Cream

| | | |
|---|---|---|
| A | Water dist. | ad 100% |
| | Glycerol | 20.0% |
| | Sodium benzoate | 2.5% |
| | Saccharin (10% strength) | 1.0% |
| | Aroma oil | 1.0% |
| | Cosmenyl Green GG | 0.005% |
| | Medialan ® LD | 6.6% |
| B | Tylose H 20 | 2.4% |
| C | Aerosil ® 300 | 2.0% |
| | Polyolefin wax 3, micronized | 32.0% |
| | Preparation: | |
| I | Mixing and stirring of components A | |
| II | Stirring of B into I | |
| III | Successive stirring of components C into II | |

### Example 11

### Hair Removal Composition

| | | |
|---|---|---|
| A | Paraffin oil | 12.0% |
| | Synthetic rubber SIS&SBS | 28.0% |
| | Piccotac 1095 | 50.0% |
| B | Polyolefin wax 4 | 10.0% |
| | Preparation: | |
| I | Melting of the components A at 200° C. and | |
| | homogenization | |
| II | Addition of B to I at 200° C. and | |
| | homogenization. Application to film strips. | |

INCI name of the commercial products used:

| | | |
|---|---|---|
| Abil B8839 | | Cyclopentasiloxane/cyclohexasiloxane |
| Abil EM90 | | Cetyldimethicone/copolyol |
| A-C 8 | (Honeywell) | Polyethylene wax |
| Aerosil ® 200 | (Degussa) | Silica gel |
| Aerosil ® 300 | (Degussa) | Silica gel |
| Aloxicoll L. | | Aluminum chlorohydrate |
| Aristoflex ® AVC | (Clariant) | Ammonium acyloyldimethyltaurate/VP |
| | | copolymer |
| Carbopol 980 | | Polyacrylate |
| Cetiol ® V | (Cognis) | Decyl oleate |
| Eutanol G | | 2-Octyldodecanol |
| Extrapon ® | (Dragoco) | Plant extracts |
| Extrapon Avocado special | | Water/ethoxydiglycol/propylene glycol/ |
| | | butylene glycol/Persea Gratissima extract |
| GENAPOL ® HS 020 | (Clariant) | Steareth-2 |
| Genapol ® HS200 | (Clariant) | Steareth-20 |
| Hostacerin ® DGI | | Polyglyceryl-2 sesquiisostearate |
| Hostapon ® KCG | | Sodium cocoyl glutamate |
| Licowax ® PE 520 | (Clariant) | Polypropylene wax from Ziegler |
| | | synthesis |
| Locron ® L | (Clariant) | Aluminum chlorohydrate |
| Medialan ® LD | (Clariant) | Sodium lauroylsarcosinate |
| Myritol ® 318 | | Capric/caprylic triglyceride |
| Nexbase ® 2004 FG | | Hydrogenated poly-1-decene |
| Nexbase ® 2006 | | Poly-1-decene |
| Nipagin ® M | (Clariant) | Methyl paraben |
| Nipaguard ® PDU | (Clariant) | Propylene glycol (and) diazolidinylurea |
| | | (and) methyl paraben (and) propyl |
| | | paraben |
| Nipasol ® M | (Clariant) | Propyl paraben |
| OCTOPIROX ® | (Clariant) | Piroctoneolamine |
| Piccotac 1095 | (Eastman) | Piperylene/butene/pentene copolymer |
| Rewoderm 66E | | Isostearate |
| SilCare ® Silicone 31M30 | (Clariant) | Caprylyl trimethicone |
| SilCare ® Silicone 31M40 | (Clariant) | Caprylyl trimethicone |
| SilCare ® Silicone 41M15 | (Clariant) | Caprylylmethicone |
| SIS&SBS | | Styrene-isoprene-styrene&styrene- |
| | | butadiene-styrene |
| Tagat R40 | | PEG-40 hydrogenated castor oil |
| Tegocare ® 450 | | Polyglyceryl-3 methylglucose |
| | | distearate |
| Tegodeo CW 90 | | Zinc ricinoleate/tetrahydroxypropyl- |
| | | ethylenediamine/laureth- |
| | | 3/propylene glycol |
| Tylose ® CB | | Carboxymethylcellulose |
| Tylose ® H 20 | | Hydroxyethylcellulose |
| Tylose ® H 4000 G4 ® | | Hydroxyethylcellulose |
| Tylose ® H 100000 YP2 | | Hydroxyethylcellulose |
| Vanclay ® | | Kaolin |
| White spirit | | Aliphatic hydrocarbon |

The following measurement methods were used:

Dropping point: DIN 51801/2

Softening point: Ring/sphere according to DIN EN 1427

Melt viscosity: DIN 53019 using a rotary viscometer

Glass transition temperature: according to DIN 51700 using differential thermoanalysis

Degree of crystallization: The degree of crystallization is calculated via the heat of fusion. This is determined using Differential Scanning Calorimetry (DSC). As a reference value for 100% crystalline polyethylene, 292 J/g is used, for ideally crystallized polypropylene 207 J/g is used.

Isotactic index: Determination through IR spectroscopy according to J. P. Luongo, J. Appl. Polm. Chem., 3, 302 (1960)

The molar mass weight average Mw, the number-average molecular weight Mn and the resulting quotient Mw/Mn were determined by gel permeation chromatography at 135° C. in 1,2-dichlorobenzene.

Paste hardness: The paste hardness indicates the mass in grams with which a punch with an area of 1 cm² has to be loaded in order to penetrate into the paste. This method is described in Seifen-Öble-Fette-Wachse, 83, p. 595 (1957).

Density (at 23° C. in g/cm³): DIN 53479

Needle penetration value (NPZ) (in 0.1 mm): DIN 51 579; DGF-M-III 9B (98); ASTM D 1321

1. A cosmetic, pharmaceutical or dermatological preparation comprising at least one wax, wherein the at least one wax is one or more homopolymer waxes of the monomers ethylene, propylene or both, one or more copolymer waxes of the monomers ethylene, propylene or both, or mixtures thereof, wherein the at least one wax has a weight-average molecular weight Mw of less than or equal to 25 000 g/mol, a number-average molecular weight Mn of less than or equal to 15 000 g/mol, a molar mass distribution Mw/Mn in the range from 1.5 to 10, wherein the at least one wax is a metallocene catalyzed wax and where wherein the one or more copolymer waxes of the monomers ethylene, propylene or both, based on the total weight of the copolymer waxes, comprise 0.1 to 30.0% by weight of structural units originating from the one monomer and 70.0 to 99.9% by weight of structural units originating from the other monomer.

2. The preparation as claimed in claim 1, wherein the at least one wax is or more homopolymer waxes of the monomer ethylene, wherein the one or more homopolymer waxes have a weight-average molecular weight Mw of less than or equal to 25 000 g/mol, a number-average molecular weight Mn of less than or equal to 15 000 g/mol and a molar mass distribution Mw/Mn in the range from 1.5 to 10.

3. The preparation as claimed in claim 1, wherein the at least one wax is one or more copolymer waxes of the monomers ethylene and propylene, wherein the one or more copolymer waxes have a weight-average molecular weight Mw of less than or equal to 25 000 g/mol, a number-average molecular weight Mn of less than or equal to 15 000 g/mol, and a molar mass distribution Mw/Mn in the range from 1.5 to 10, and wherein the one or more copolymer waxes, based on the total weight of the one or more copolymer waxes, comprise 70.0 to 99.9% by weight of structural units originating from the monomer ethylene and 0.1 to 30.0% by weight of structural units originating from the monomer propylene.

4. The preparation as claimed in claim 1, wherein the at least one wax is one or more homopolymer waxes of the monomer propylene, wherein the one or more homopolymer waxes have a weight-average molecular weight Mw of less than or equal to 25 000 g/mol, a number-average molecular weight Mn of less than or equal to 15 000 g/mol, a molar mass distribution Mw/Mn in the range from 1.5 to 10.

5. The preparation as claimed in claim 1, wherein the at least one wax is one or more copolymer waxes of the monomers ethylene and propylene, wherein the one or more copolymer waxes have a weight-average molecular weight Mw of less than or equal to 25 000 g/mol, a number-average molecular weight Mn of less than or equal to 15 000 g/mol, a molar mass distribution Mw/Mn in the range from 1.5 to 10 and where the one or more copolymer waxes, based on the total weight of the one or more copolymer waxes, comprise 0.1 to 30.0% by weight of structural units originating from the monomer ethylene and 70.0 to 99.9% by weight of structural units originating from the monomer propylene.

6. The preparation as claimed in claim 1, wherein the at least one wax has a ring/sphere dropping or softening point between 80 and 165° C. and a melt viscosity, measured at a temperature of 170° C., between 20 and 40 000 mPa s.

7. The preparation as claimed in claim 1, wherein the one or more copolymer waxes of the monomers ethylene propylene or both have a glass transition temperature of at most −20° C.

8. The preparation as claimed in claim 1, wherein the at least one wax has a degree of crystallization of more than 55%.

9. The preparation as claimed in claim 1, wherein the at least one wax has a degree of crystallization below 55%.

10. The preparation as claimed in claim 1, wherein the at least one wax further comprises waxes which are not polar modified, and one or more waxes which are polar modified.

11. The preparation as claimed in claim 1, wherein the at least one wax is chosen from non-polar modified waxes.

12. The preparation as claimed in claim 1, wherein the at least one wax is chosen from polar modified waxes.

13. The preparation as claimed in claim 1, wherein the preparation is in the form of a pencil, cover stick, acne stick, lipstick, a make-up, a foundation, a face powder, blusher, a mascara, an eyeshadow, eyeliner, a peel cream, a hair wax, hair styling composition, styling fluid, a hair foam, a hair gel, hair spray, a mousse, a hair oil, an end fluid, a hair treatment, night cream, care cream, nutrient cream, perfume cream, body lotion, ointment, a lipcare composition, sunscreen composition, deodorant, antiperspirant, colored gel in the form of a pencil, such as, for example, a multiphase pencil, a stick, a paste, a powder, a cream, a cream foam, a lotion, a self-foaming emulsion, foam-like emulsion, after-foaming emulsion, foamable emulsion, a gel, roll-on preparation, a foam or a depilatory.

14. The preparation as claimed in claim 1, wherein the preparation is the form of a stick or pencil.

15. The preparation as claimed in claim 1, wherein the preparation is in the form of an emulsion.

16. The preparation as claimed in claim 15, which is in the form of an oil-in-water emulsion.

17. The preparation as claimed in claim 16, wherein, based on the total weight of the preparation, comprises
a) up to 95% by weight of a water phase,
b) up to 40% by weight of an oil phase,
c) up to 15% by weight of one or more emulsifiers and
d) up to 5% by weight of the at least one wax.

18. The preparation as claimed in claim 16, which is in the form of a gel cream of the oil-in-water type, and, based on the total weight of the preparation, comprises
a) up to 95% by weight of a water phase,
b) up to 30% by weight of an oil phase,
c) up to 5% by weight of one or more emulsifiers and
d) up to 5% by weight of the at least one wax.

19. The preparation as claimed in claim 15, which is in the form of a water-in-oil emulsion.

20. The preparation as claimed in claim 19, which based on the total weight of the preparation, comprises
a) up to 95% by weight of a water phase,
b) up to 60% by weight of an oil phase,
c) up to 20% by weight of one or more emulsifiers and
d) up to 5% by weight wax of the at least one wax.

21. The preparation as claimed in claim 1, wherein the at least one wax is in micronized form.

22. The preparation as claimed in claim 1, which is in the form of a dispersion and comprises
a) a carrier material,
b) one or more emulsifiers and
c) optionally, one or more waxes in addition to the at least one wax.

23. The preparation as claimed in claim 1, wherein the preparation is in the form of a decorative composition.

24. The preparation as claimed in claim 1, wherein the preparation is in the form of a powder, compact, a paste, cream or a stick, and wherein the preparation further comprises one or more colorants.

25. The preparation as claimed in claim 1, wherein the preparation is in the form of a sunscreen composition and further comprises one or more UV photoprotective filters.

26. The preparation as claimed in claim 1, further comprising one or more antioxidants.

27. The preparation as claimed in claim 1, wherein the preparation further comprises one or more substances selected from the group consisting of antimicrobially effective substances, astringents and deodorizing substances, wherein the preparation is in the form of a deodorant or antiperspirant and wherein the deodorant or antiperspirant is in the form of a spray, stick, a paste, a gel or a lotion.

28. The preparation as claimed in claim 27 wherein the deodorizing substances are allantoin bisabolol.

29. The preparation as claimed in claim 1, wherein the preparation is in the form of a peel.

30. The preparation as claimed in claim 1, wherein the preparation is in the form of a dental care composition.

31. The preparation as claimed in claim 1, wherein the preparation is in the form of a hair removal composition.

32. The preparation as claimed in claim 1, wherein the at least one wax has a molar mass distribution Mw/Mn in the range from 1.5 to 5.

33. The preparation as claimed in claim 1, wherein the at least one wax has a molar mass distribution Mw/Mn in the range 1.5 to 3.

34. The preparation as claimed in claim 1, wherein the at least one wax has a molar mass distribution Mw/Mn in the range from 2 to 2.5.

35. The preparation as claimed in claim 2, wherein the one or more homopolymer waxes have a weight-average molecular weight Mw of 1000 to 22 000 g/mol, a number-average molecular weight Mn of 500 to 12 000 g/mol and a molar mass distribution Mw/Mn in the range from 1.5 to 5.

36. The preparation as claimed in claim 2, wherein the one or more homopolymer waxes have a weight-average molecular weight Mw of 4000 to 20 000 g/mol, a number-average molecular weight Mn of 1000 to 5000 g/mol and a molar mass distribution Mw/Mn in the range from 1.5 to 3.

37. The preparation as claimed in claim 2, wherein the one or more homopolymer waxes have a molar mass distribution Mw/Mn in the range from 2 to 2.5.

38. The preparation as claimed in claim 3, wherein the one or more copolymer waxes have a weight-average molecular weight Mw of 2000 to 22 000 g/mol, a number-average molecular weight Mn of 1000 to 12 000 g/mol and a molar mass distribution Mw/Mn in the range from 1.5 to 5.

39. The preparation as claimed in claim 3, wherein the one or more copolymer waxes have a weight-average molecular weight Mw of 4000 to 20 000 g/mol, a number-average molecular weight Mn of 2000 to 10 000 g/mol and a molar mass distribution Mw/Mn in the range from 1.5 to 3.

40. The preparation as claimed in claim 3, wherein the one or more copolymer waxes have a molar mass distribution Mw/Mn in the range from 2 to 2.5.

41. The preparation as claimed in claim 4, wherein the one or more homopolymer waxes have a weight-average molecular weight Mw of I of 2000 to 22 000 g/mol, a number-average molecular weight Mn of 1000 to 12 000 g/mol, a molar mass distribution Mw/Mn in the range from 1.5 to 5 and have an isotactic index of 75 to 95%.

42. The preparation as claimed in claim 4, wherein the one or more homopolymer waxes have a weight-average molecular weight Mw of 4000 to 22 000 g/mol, a number-average molecular weight Mn of 2000 to 10 000 g/mol, a molar mass distribution Mw/Mn in the range from 1.5 to 3 and have an isotactic index of 80 to 90%.

43. The preparation as claimed in claim 4, wherein the one or more homopolymer waxes have a molar mass distribution Mw/Mn in the range from 2 to 2.5.

44. The preparation as claimed in claim 5, wherein the one or more copolymer waxes have a weight-average molecular weight Mw of 2000 to 22 000, a number-average molecular weight Mn of 1000 to 12 000 g/mol, a molar mass distribution Mw/Mn in the range from 1.5 to 5.

45. The preparation as claimed in claim 5, wherein the one or more copolymer waxes have a weight-average molecular weight Mw of 4000 to 20 000 g/mol, a number-average molecular weight Mn of 2000 to 10 000 g/mol and a molar mass distribution Mw/Mn in the range from 1.5 to 3.

46. The preparation as claimed in claim 5, wherein the one or more copolymer waxes have a molar mass distribution Mw/Mn in the range from 2 to 2.5.

47. The preparation as claimed in claim 1, wherein the at least one wax has a degree of crystallization of between 60 and 90%.

48. The preparation as claimed in claim 1, wherein the at least one wax has a degree of crystallization below 50%.

49. The preparation as claimed in claim 17, comprising:
a) 70 to 90% by weight of a water phase,
b) 1 to 40% by weight of an oil phase,
c) 0.5 to 12% by weight of one or more emulsifiers and
d) 0.01 to 5% by weight of the at least one wax.

50. The preparation as claimed in claim 17, comprising:
a) 70 to 90% by weight of a water phase,
b) 2 to 25% by weight of an oil phase,
c) 1 to 8% by weight of one or more emulsifiers and
d) 0.05 to 3% by weight of the at least one wax.

51. The preparation as claimed in claim 17, comprising:
a) 75 to 85% by weight of a water phase,
b) 5 to 20% by weight of an oil phase,
c) 1 to 5% by weight of one or more emulsifiers and
d) 0.1 to 2% by weight of the at least one wax.

52. The preparation as claimed in claim 16, comprising:
a) 50 to 95% by weight of a water phase,
b) 1 to 30% by weight of an oil phase,
c) 0.5 to 5% by weight of one or more emulsifiers and
d) 0.01 to 5% by weight of the at least one wax.

53. The preparation as claimed in claim 16, comprising:
a) 70 to 90% by weight of a water phase,
b) 3 to 25% by weight of an oil phase,
c) 0.2 to 4% by weight of one or more emulsifiers and
d) 0.05 to 3% by weight of the at least one wax.

54. The preparation as claimed in claim 16, comprising:
a) 75 to 85% by weight of a water phase,
b) 5 to 15% by weight of an oil phase,
c) 0.5 to 3% by weight of one or more emulsifiers and
d) 0.1 to 2% by weight of the at least one wax.

55. The preparation as claimed in claim 19, which, based on the total weight of the preparation, comprises:
a) 40 to 95% by weight of a water phase,
b) 2 to 60% by weight of an oil phase,
c) 0.5 to 20% by weight of one or more emulsifiers and
d) 0.01 to 5% by weight of the at least one wax.

56. The preparation as claimed in claim 19, which, based on the total weight of the preparation, comprises:
a) 50 to 90% by weight of a water phase,
b) 5 to 40% by weight of an oil phase,
c) 1 to 15% by weight of one or more emulsifiers and
d) 0.05 to 3% by weight of the at least one wax.

57. The preparation as claimed in claim 19, which, based on the total weight of the preparation, comprises
a) 60 to 85% by weight of a water phase,
b) 10 to 30% by weight of an oil phase,
c) 4 to 12% by weight of one or more emulsifiers and
d) 0.1 to 2% by weight of the at least one wax.

58. The preparation as claimed in claim 22, wherein the carrier material is one or more oil components, one or more sovents, or a mixture thereof.

59. The preparation as claimed in claim 24, wherein the one or more colorants is selected from the group of color lakes, toners and pigments.

60. The preparation as claimed in claim 25, wherein the sunscreen composition is in the form of a spray, stick, a paste, a gel or a lotion.
